(19) United States
(12) Patent Application Publication
(10) Pub. No.: US 2005/0181991 A1
(43) Pub. Date: Aug. 18, 2005

(54) Use of artemin, a member of the GDNF ligand family

(76) Inventors: David L. Shelton, Oakland, CA (US); Heidi S. Phillips, Palo Alto, CA (US)

Correspondence Address: HELLER EHRMAN LLP, 275 MIDDLEFIELD ROAD, MENLO PARK, CA, 94025-3506, US

(21) Appl. No.: 10/794,801

(22) Filed: Mar. 5, 2004

Related U.S. Application Data
(62) Division of application No. 10/451,567, filed on May 11, 2004.
(60) Provisional application No. 60/257,601, filed on Dec. 22, 2000.

(51) Int. Cl. A61K 38/18

(52) U.S. Cl. 514/12

(57) ABSTRACT

The present invention concerns the use of artemin for the prevention or treatment of nerve cell injury and changes associated with nerve cell injury. More particularly the present invention provides for a method of protecting neurons in a mammal from injury-induced pathological changes and a method of treating neuronal damage in a mammal by administering artemin or an artemin agonist.

### FIELD OF THE INVENTION

The present invention concerns the use of artemin for the prevention, amelioration or treatment of nerve cell injury and changes associated with nerve cell injury.

### BACKGROUND OF THE INVENTION

Artemin is a recently identified and characterized neurotrophic factor. Artemin is a member of the glial cell line-derived neurotrophic factor (GDNF) family and appears to signal through the GFR-3 receptor (Baloh et al., Neuron, 21:1291-1302 (1998)). Like all known members of the GDNF family, artemin has been found to support the survival of both dopaminergic midbrain neurons and peripheral neurons in vitro (Baloh et al., supra). In addition, artemin also appears to protect nigrostriatal dopaminergic neurons in vivo (Rosenblad et al., Mol. Cell. Neurosci., 15:199-214 (2000)).

Protein neurotrophic factors or neurotrophins influence growth and development of the vertebrate nervous system. In addition, they are believed to play an important role in promoting the differentiation, survival, and function of a diverse group of neurons in the brain and periphery. A number of neurotrophic factors have been proposed as a potential means for enhancing specific neuronal cell survival. Thus it has been suggested that they may be useful in the treatment of neurodegenerative diseases such as amyotrophic lateral sclerosis (ALS), Alzheimer's disease, stroke, epilepsy, Huntington's disease, Parkinson's disease and peripheral neuropathy.

Neurotrophic factors are believed to have important signaling functions in neural tissues, based in part upon the precedent established with nerve growth factor (NGF). NGF supports the survival of sympathetic, sensory, and basal forebrain neurons of developing animals both in vitro and in vivo. Administration of exogenous NGF rescues neurons from cell death during development. Conversely, removal or sequestration of endogenous NGF by administration of anti-NGF antibodies promotes such cell death (Heumann, J. Exp. Biol., 132:133-150 (1987); Hefti, J. Neurosci., 6:2155-2162 (1986); Thoenen et al., Annu. Rev. Physiol., 60:284-335 (1980)).

Based on its neurotrophic properties, NGF has been the subject of a great deal of research. (For review see Hefti et al., Neurobiol. Aging, 10:515-533 (1988) and Levi-Montalcini, Science, 237:1154-1164 (1987)). However, in addition to its neuroprotective and neuroregenerative effects, NGF also produces hyperalgesia and pain in both animals and humans following intravenous (iv), subcutaneous (sq) or intradermal (id) injection or intracerebroventricular (icv) or intrathecal (it) administration (Lewin et al., J. Neurosci., 13:2136-2148 (1993), Lewin et al., Eur. J. Neurosci. 6:1903-1912 (1994), Andreev et al., Pain, 63:109-115 (1995); Petty et al., Ann. Neurol., 36:244-246 (1994); Hao et al., Neurosci, Lett., 286:208-212 (2000)). This finding has been born out in clinical trials in humans where the pain associated with NGF treatment has limited its clinical efficacy. See, e.g. Eriksdotter et al., Demem. Genisis. Cogs. Disord. 9:246-257 (1998) Pony et al., supra, and Apfel et al., JAMA 284:2215-2221 (2000).

Many of the small diameter sensory neurons in the dorsal root ganglia (DRG) express high affinity NGF receptors (Verge et al., J. Neurocystal, 18:583-591 (1989). McMahon et al., Neurol. 12:1161-1171 (1994)) and these NGF receptor expressing neurons largely also express sensory neuropeptide, especially P and CGRP (Verge et al., supra, and Averill et al., Eur. J. Neurosci., 7:1484-1494 (1995)). Consistent with the localization of NGF recptors on these peptidernic neurons, it has been shown that treatment with NGF can modify the expression of these peptides within these neurons. Treatment with NGF can induce supranormal levels of the peptides CGRP and substance P in vivo (Goadart et al., Proc. Nat. Acad. Sci., 78:5895-5898 (1981) and Amann et al., Neurosci. Let., 203:171-174 (1996)). Experiments examining the effect of NGF on DRG neurons grown in vitro have shown that this is a direct effect (Lindsay and Mamar, Nature, 337:382-384 (1989)). Moreover, NGF treatment causes increased release of substance P and CGRP from the sesory neuron projection to the dorsal horn (Malcangie et al., Eur. J. Neurosci. 9:1101-1104(1997) and Malcagie et al., Eur. J. Neurosci., 12:139-144 (2000)).

Injury to these peptide-containing neurons has been shown to lead to a deficit in peptide content, not only in the neuronal cell bodies, but also in their peripheral projections and their projection into the dorsal horn of the spinal cord. For example, sciatic nerve transaction leads to a decrease in substance P content both in the DRG and in the dorsal horn of the spinal cord in the area corresponding to the sciatic nerve projection. NGF administration after injury has been demonstrated to prevent this injury induced change in peptide content in the DRG (Verge et al. J. Neurosci. 15:2081-2098 (1995)) and in the spinal cord dorsal horn projection. (Bennett et al., Med. Cell Neurosci., 8:211-220 (1998)). Injury to these neurons may also be caused by chemical (chemotherapeutic agents or capsaicin) or metabolic (diabetes) insults, and in all of these cases the peptide content of the nerons decrease upon injury and NGF treatment is able to reverse or prevent this decrease (Apfel, et al., Ann. Neurol., 29:87-89 (1991), Apfel. et al., Ann. Neurol., 31:78-80 (1992) Donneror, et al., Brain Res., 741:103-108 (1996) and Apfel et al., Brain Res., 634:7-12 (1994)).

The regulation of substance P by NGF is of interest because substance P has long been associated with pain. Indeed, administration of substance P either peripherally or centrally causes an increase in pain and pain associated behaviors in both man and experimental animals (Masda, et al., J. Neurol Transmission, 96:125-133 (1994). Hong et al., Neuroscience, 63:827-838 (1994) Dirip and Yaksh, Neurosci. Let., 220:93-96 (1996), Heppelmann and Pawlak, Neurosci. Let., 223:97-100 (1997), Holthuson et al., Neuropeptides, 31:445-448 (1997), Yoast et al., Life Sciences, 62:319-325 (1998), Hitosugi et at., Mol. Find. Exp. Clin. Pharm., 21:409-413 (1999)). Consequently, the increase in substance P, and the increase in the release of substance P, that is caused by NGF has been proposed to be responsible for the pain and hyperalgesia associated with NGF administration. Consistent with this view, the pain associated with increased NGF can be blocked by antagonists of substance P action. For example, mice that over-express NGF exhibit overgrowth of substance P fibers in the dorsal horn and show hyperalgesia and allodyria. These pain behaviors can be prevented with substance P recptor antagonists (Ma et al., Eur. J. Neurosci., 7:2021-2035 (1995)). In addition, Hao et al. have reported that the mechanical and heat hyperalgesia that is present in rats after NGF treatment can be alleviated by antagonists that are selective to the substance P receptor NGF (Neuroscience Lett., 286:208-212 (2000)).

Thus there is a need in the art for a method of effectively treating nerve damage and neurological disorders without producing the side effects that make NGF treatment impractical. Especially, there is need for an effective treatment for peptidergic sensory neurons. These cells decrease their expression of peptide as a result of injury and treatment that protects them from, or repairs them after injury must also increase their peptide expression. However this increase in peptide expression is predicted to lead to adverse events such as pain.

Several additional neurotrophic factors related to NGF have been identified and characterized. These are not members of the so called neurotrophin family and include brain-derived neurotrophic factor (BDNF) (Leibrock, et al., Nature, 341:149-152(1989)), neurotrophin-3 (NT-3) (Kaisho, et al., FEBS Lett., 266:187 (1990); Maisonpierre, et al., Science, 247:1446 (1990); Rosenthal, et al., Neuron, 4:767 (1990)), neurotrophin 4/5 (NT4/5) (Berkemeier, et al., Neuron, 7:857-866 (1991)). Also identified but belonging to a separate family of molecules is glial cell line-derivative neurotrophic factor (GDNF; Lin et al., Science, 260:1130-1132 (1993)). Similarly to NGF, GDNF administration has been associated with weight loss and allodynia (Hoane et al., Exp. Neurol., 160:235-243 (1999)).

The identified neurotrophic factors can be grouped in families based on their amino acid and structure similarities. The GDNF family was found to include not only GDNF, but also the related molecules neurturin and persephin (Lin et al., Science, 260:1130-1132 (1993); Henderson et al., Science, 266:1062-1064 (1994); Buj-Bello et al., Neuron, 15:821-828 (1995); Kotzbauer et al., Nature, 384:467-470 (1996); Milbrandt et al., Neuron, 20:245-2 (1998)).

GDNF, neurturin and persephin have all been found to support the survival of dopaminergic midbrain neuron in vivo and in vitro (Lin et al., Science, 260:1130-1132 (1993); Henderson et al., Science, 266:1062-1064 (1999Horger et al., J. Neurosci., 18:4929-4937 (1998); Oppenheim et al., Nature, 373:344-346 (1995); Milbrandt et al., Neuron, 20:245-253 (1998)). This ability has suggested that these compounds might be useful in treating disorders whose basis is neuronal damage or degeneration. Interestingly, while GDNF and neurturin are also able to increase the survival of several types of peripheral neurons in culture, the same has not been shown to be true of persephin (Buj-Bello et al., Neuron, 15:821-828 (1995); Kotzbauer et al., Nature, 384:467-470 (1996); Ebendal et al., Neurosci., Res. 40:276-284 (1995); Heuckeroth et al., Dev. Biol, 200:116-129 (1998); Trupp et al., J. Cell Biol. 130:137-148 (1995); Milbrandt et al., Neuron, 20:245-253 (1998)).

All members of the GDNF family of neurotrophic factors appear to signal through a receptor complex that includes a PI-linked ligand binding protein called GFR and a transmembrane signaling protein that is a protein-tyros kinase called RET. While RET seems to be a common signaling element for all members of the GDNF neurotrop factor family, there are four distinct GFR s (GFR 1-4) each of which appears to be relatively specific for a different ligand. GFR 1-RET preferentially binds GDNF while GFR 2-RET preferentially binds neurturin (Baloh et al., Neur. 18:793-802 (1997); Jing et al., Cell, 85:1113-1124(1996); Klein et al., Nature, 387:717-721(1997); Sanicola et al., Proc. Natl. Acad. Sci. USA, 94:6238-6243 (1997); Suvanto et al., Hum. Molec. Genetics, 6:1267-1273 (1997); Traeanor et al., Nature, 382:80-83 (1996)). However, recently there has been some evidence that this apparent specificity of GFR 1 for GDNF and GFR 2 for neurturin may be somewhat oversimplified (Wang et al., J. Neurosci. Res., 61:1-9 (2000)). In addition there has been evidence published which indicates that the GFR 1 receptor may be able to transduce a signal from GDNF even in the absence of RET (Trupp et al., J. Biol Chem., 274:20885-20894 (1999)). Persephin has been reported to bind to GFR 4, and thus is likely to act through GFR 4-RET (Enokido et al., Curr. Biol., 8:1019-1022 (1998)). Analysis of GFR 3, on the other hand, indicated that it could not form a functional receptor with RET capable of being activated by GDNF, neurturin or persephin (Baloh et al., Proc. Natl. Acad. Sci., USA 95:5801-5806 (1998)). This suggested that a fourth member of the GDNF family remained to be identified.

The fourth member of the GDNF family has recently been identified and characterized and is called artemin (Baloh et al., Neuron, 21:1291-1302 (1998)). Several other factors that are identical or nearly identical to artemin have also been identified and called enovin (Masure et al., Eur. J. Biochem., 266:892-902 (1999)) and neublastin (Rosenblad et al., Mol. Cell Neurosci., 15:199-214 (2000)). The predicted full-length arternin protein contains a signal peptide for secretion and a proregion that is separated from the mature region by several conserved RXXR furin protease cleavage sites. In addition, the human artemin gene contains two possible starting methionines, leading to the alternative production of two different full-length human artemin polypeptides. However, the mature form of human artemin resulting from cleavage of the prdregion is identical in both forms. The mouse. artemin gene does not contain an alternative methionine. Artemin is more similar at the amino acid level to neurturin and persephin (45% identity) than to GDNF (36% identity) (Baloh et al., supra).

As mentioned briefly above, Baloh et al., supra, have reported that artemin supports the survival of dopaminergic midbrain neurons in culture. Artemin has also been found to protect nigrostriatal dopaminergic neurons in vivo (Rosenblad, supra). Additionally, like GDNF and neurturin, artemin is able to support peripheral neurons in vitro, including a subset of sensory neurons from both the dorsal root ganglion (DRG) and the trigeminal ganglion (TG), visceral sensory neurons from the nodose ganglion (NG). and sympathetic neurons of the superior cervical ganglia (SCG) (Baloh et al., supra).

Artemin has been reported to be able to bind GFR 3 and to activate GFR 3-RET receptor complexes (Baloh et al. supra). Consistently, of the four GDNF family neurotrophins, artemin is expressed in a pattern that is most consistent with that of the GFR 3 receptor (Baloh et al., supra). Similar binding and expression results were obtained with the identical molecule enovin (Masure at al, supra). Thus it appears that GFR 3 is the functional receptor for artemin.

Problems with the development and. survival of neurons are a major contributor to many neurological disorders, ranging from acute injury to long-term degeneration. Thus it is an aim of the present invention to provide a means of using artemin to prevent neuronal death and increase neuronal survival without the accompanying deleterious side effects seen with NGF treatment.

### BRIEF SUMMARY OF THE INVENTION

The present invention is based on the finding that artemin is highly efficient in the prevention, amelioration and treatment of injury-induced changes in neurons, particularly small sensory neurons, in mammals, and, in fact, protects peptidergic neurons from the loss of substance P after injury. Although artemin administration can increase the content of peptide similarly to NGF, it is unexpectedly devoid of the deleterious side effects, in particular, hyperalgesia, often associated with the administration of other neurotrophic factors, such as NGF.

In one aspect, the present invention provides a method of protecting neurons in a mammal from injury induced pathological changes. The method comprises administering to the mammal artemin or an artemin agonist. The mammal to which artemin is administered may be human. In a specific embodiment, the dosage of artemin or artemin agonist is between about 0.01 g/kg and about 1 mg/kg. The administration of the artemin or artemin agonist is not accompanied by mechanical or thermal hyperalgesia.

In one embodiment the dose is between about 0.1 g/kg and about 1 mg/kg. In an alternate embodiment, the dose is between about 0.1 mg/kg and about 1 mg/kg.

Administration may be by any method known to those of skill in the art. In a preferred embodiment, the administration is systemic and may be by intravenous, intradermal, intrathecal or subcutaneous injection. In an alternate embodiment, the administration may be topical.

The administration of artemin or an artemin agonist is repeated as determined by methods known in the art. In one embodiment, the administration is repeated at least two times a week for a duration of at least two weeks. In another embodiment the administration is repeated at least three times a week for a duration of at least two weeks.

In one embodiment of the invention, the neurons which are affected by artemin are peripheral neurons. More preferably, the neurons are selected from the group consisting of sympathetic, parasympathetic, sensory and enteric neurons. Even more preferably the neurons are small fiber sensory neurons.

In yet another embodiment of the invention, the neurons are motor neurons or central neurons. When the neurons are central neurons they may preferably be brain or spinal cord neurons.

The injury contemplated in the first aspect of the invention may be associated with trauma, a toxic agent, adverse side effects of other therapeutic agents, surgery, stroke, ischemia, infection, metabolic disease, nutritional deficiency or malignancy. The injury may also be associated with peripheral neuropathy, more preferably a peripheral sensory neuropathy and even more preferably diabetic neuropathy.

The artemin used in the methods of the invention may be native sequence artemin polypeptide or native sequence human artemin polypeptide. In one embodiment the native sequence human artemin polypeptide comprises the amino acid sequence of SEQ ID NO: 1. The native sequence human artemin polypeptide may also comprise the amino acid sequence of either SEQ ID NO: 3 or SEQ ID NO: 5.

Another aspect of the invention is a method of treating neuronal damage in a mammal. The method comprises administering artemin or an artemin agonist to the mammal at a dose of between about 0.01 g/kg and about 1 mg/kg. In alternate embodiments, the dose is between about 0.1 g/kg and about 1 mg/kg or between about 0.1 mg/kg and about 1 mg/kg. The administration is not accompanied by mechanical or thermal hyperalgesia. The mammal to which artemin is administered may be human.

The neuronal damage contemplated in this aspect of the invention may result from a neuropathy or neurodegenerative disease. In one embodiment the neuropathy is peripheral neuropathy and in another embodiment it is diabetic neuropathy. In yet another embodiment the neuropathy is smallfiber sensory neuropathy. More particularly the neurodegenerative disease may be amyotrophic lateral sclerosis.

In one embodiment a native sequence artemin polypeptide is administered. In another embodiment a native sequence human artemin polypeptide is administered. The native sequence human artemin polypeptide may comprise the amino acid sequence of SEQ ID NO: 1. In addition, the native sequence human artemin polypeptide may comprise the amino acid sequence of either SEQ ID NO: 3 or SEQ ID NO: 5. Administration of the artemin may be systemic or topical and preferably is repeated at least two times a week for a duration of two weeks.

A third aspect of the present invention is an article of manufacture that comprises a container, a pharmaceutical composition comprising artemin within the container and instructions to administer the pharmaceutical composition at a dose which is between about 0.01 g/kg and about 1 mg/kg.

### BRIEF DESCRIPTION OF THE DRAWINGS

FIG. 1 depicts the amino acid sequence of mature human artemin (SEQ ID NO: 1).

FIG. 2 depicts a polynucleotide sequence of one form of full-length human artemin (SEQ ID NO: 2).

FIG. 3 depicts the amino acid sequence (SEQ ID NO: 3) of the form of human artemin that is encoded by the polynucleotide sequence of FIG. 2.

FIG. 4 depicts a polynucleotide sequence that encodes a second variant of human artemin (SEQ ID NO: 4).

FIG. 5 depicts the amino acid sequence (SEQ ID NO: 5) of the variant of human artemin that is encoded by the polynucleotide sequence of FIG. 4.

FIG. 6 depicts a polynucleotide sequence encoding full-length artemin derived from the mouse (SEQ ID NO: 6).

FIG. 7 depicts the amino acid sequence (SEQ ID NO: 7) of mouse artemin encoded by the polynucleotide sequence of FIG. 6.

FIG. 8 depicts thermal withdrawal latencies in animals treated systemically with NGF and artemin. In NGF treated animals, thermal withdrawal latencies dropped to approximately 75% of the pretreatment value while latencies in animals treated with artemin did not.

FIG. 9 depicts tests of mechanical sensitivity. Animals treated systemically with NGF had their withdrawal thresholds drop to approximately 60% of their pretreatment level, while animals treated with either saline or artemin gradually dropped to about 90% of their pretreatment score.

FIG. 10 depicts thermal withdrawal latencies in animals treated locally with NGF and artemin. Thermal withdrawal latencies decreased in animals treated with NGF but stayed at their pretreatment levels in animals treated with artemin.

FIG. 11 depicts tests of mechanical sensitivity. In animals treated locally with NGF, withdrawal thresholds drop to approximately 22 grams from a pretreatment value of 27 grams, while the withdrawal threshold did not drop appreciably in animals treated locally with either saline or artemin.

FIG. 12 shows that animals treated with NGF lost approximately 7% of their body mass in two weeks while animals treated with either artemin or saline lost less than 2% of their body mass.

FIG. 13 shows that constant treatment with NGF increased the frequency of pain behaviors in rats with spared nerve injury while treatment with artemin decreased the incidence of pain behaviors.

FIG. 14 shows that both NGF and artemin protect neurons from herpes simplex virus induced death.

FIG. 15 shows that artemin treatment abolishes the decrease in peptide content of small sensory neuron seen after sciatic nerve transection.

FIG. 16 shows the method of quantifying peptidergic content. By calculating average intensity of staining along each line, the variation in staining intensity with depth down the dorsal horn was calculated.

FIG. 17 depicts the intensity of substance P staining plotted against depth from the surface of the dorsal horn for rats with sciatic nerve injury, artemin prevents axotomy-induced peptide loss.

FIG. 18 shows that artemin protects neurons from an axotomy induced shift in C fiber conduction velocity

FIG. 19 shows that the survival enhancing effect of artemin on neonatal neuron survival requires the function of GFRα3. In neurons from GFRα3 knockout mice, addition of artemin caused no increased survival over that seen in controls.

FIG. 20 shows that peripheral nerve injury induces an upregulation in GFRa3 expression so that almost a small DRG cells express the receptor. A concomitant upregulation of artemin is seen in the distal stump.

FIG. 21 shows that capsaicin treatment causes a profound deficit in the substance P content of the medial and lateral dorsal horn. Artemin administration completely protects the peptide content of the sensory neuro projection into the dorsal horn.

FIG. 22 shows that artemin infusion into the cerebral ventricle prevents the loss of substance associated with sciatic nerve transection.

### DETAILED DESCRIPTION OF THE INVENTION

A. Definitions

As used herein, the terms “artemin” and “artemin polypeptide,” which are used interchangeably, refer to native sequence artemin, artemin variants, and chimeric artemin, each of which is defined herein. Optionally, the artemin is not associated with native glycosylation. “Native glycosylation” refers to the carbohydrate moieties that are covalently attached to artemin when it is produced in mammalian cells, particularly in the cells in which it is produced in nature. Accordingly, human artemin produced in a non-human cell is an example of artemin which may “not be associated with native glycosylation.” Sometimes the artemin may not be glycosylated at all, as in the case where it is produced in prokaryotes, e.g. E. coli.

Artemin nucleic acid is RNA or DNA which encodes an artemin polypeptide, as defined above, or which hybridizes to such DNA or RNA and remains stably bound to it under stringent hybridization conditions and is greater than about 10 nucleotides in length. Stringent conditions are those which (1) employ low ionic strength and high temperature for washing, for example, 0.15 M NaCl/0.015 M sodium citrate/0.1% NaDodSO₄ at 50° C., or (2) use during hybridization a denaturing agent such as formamide, for example, 50% (vol/vol) formamide with 0.1% bovine serum albumin/0.1% Ficoll/0.1 % polyvinlypyrrolidone/50 mM sodium phosphate buffer at pH 6.5 with 750 mM NaCl, 75 mM sodium citrate at 42° C.

Nucleic acid is operably linked when it is placed into a functional relationship with another nucleic acid sequence. Artemin nucleic acid may be operably linked with another nucleic acid sequence in a vector such that it may be expressed in a particular host organism. This may be done by methods well known in the art. For example, DNA for a presequence or a secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contiguous and in reading frame. However, enhancers do not have to be contiguous. Linking is accomplished by ligation at convenient restriction sites. If such sites do not exist, then synthetic oligonucleotide adapters or linkers are used in accord with conventional practice.

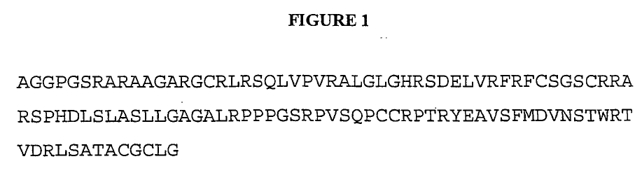

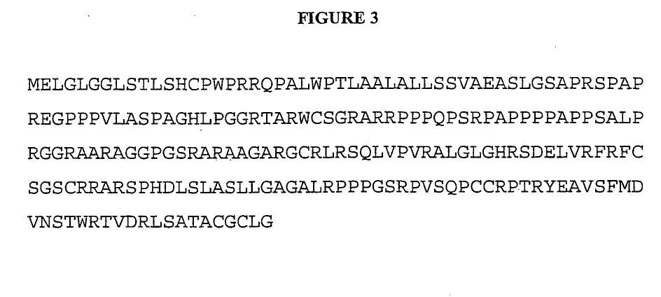

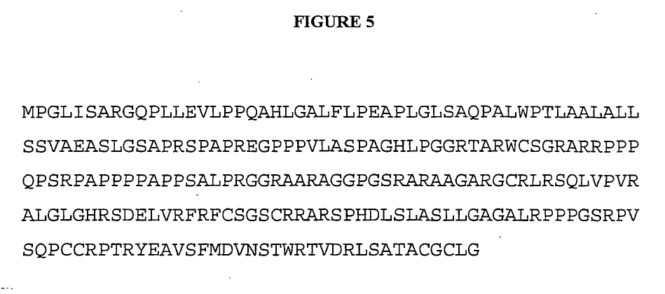

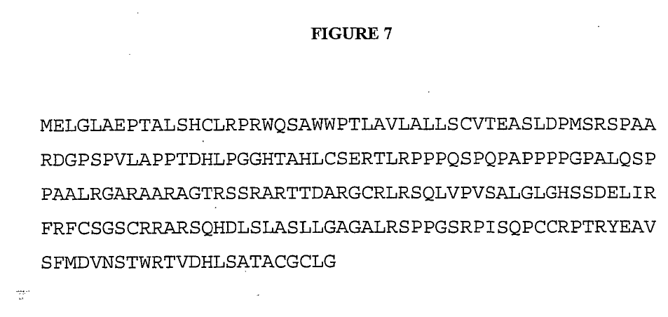

A “native sequence artemin” comprises a polypeptide having the same amino acid sequence as artemin derived from nature, regardless of its mode of preparation. Thus, a native sequence artemin can have the amino acid sequence of naturally occurring rat artemin, murine artemin, human artemin, or artemin from any other mammalian species. For example, two native sequence full-length human artemin amino acid sequences are presented in FIGS. 3 and 5 (SEQ ID NOS: 3 and 5). These two sequences are the result of the presence of two possible starting methionines in the human artemin gene. A native sequence mouse artemin amino acid sequence is presented in FIG. 7 (SEQ ID NO: 7). Such native sequence artemin polypeptides can be isolated from nature or produced by recombinant and/or synthetic means. The term “native sequence artemin” specifically encompasses naturally occurring prepro, pro and mature forms and truncated forms of artemin, naturally occurring variant forms (e.g. alternatively spliced forms), and naturally occurring allelic variants. The preferred native sequence artemin is a mature human native sequence artemin as shown in FIG. 1 (SEQ ID NO: 1). The mature form of human artemin is the product of proteolytic cleavage of a large proregion found in each of the two fulllength native sequences.

“Artemin variants” are biologically active artemin polypeptides having an amino acid sequence which differs from the sequence of a native sequence artemin polypeptide, such as that shown in FIG. 1 for mature human artemin, by virtue of an insertion, deletion, modification andlor substitution of one or more amino acid residues within the native sequence, such as the sequence of FIG. 1 (SED ID NO: 1). Artemin variants generally have less than 100% sequence identity with a native sequence artemin such as the mature human artemin of FIG. 1 (SEG ID NO: 1). Ordinarily, however, a biologically active artemin variant will have an amino acid sequence with at least about 60% amino acid sequence identity with the amino acid sequence of a naturally occurring artemin such as the mature human artemin of FIG. 1 (SEQ ID NO: 1), preferably at least about 65%, 70%, 75%, 80%, with increasing preference of at least about 85% to at least about 99% amino acid sequence identity, in 1% increments. The artemin variants include peptide fragments of at least 5 amino acids, preferably at least 10 amino acids, more preferably at least 15 amino acids, even more preferably at least 20 amino acids that retain a biological activity of the corresponding native sequence artemin polypeptide. Artemin variants also include artemin polypeptides wherein one or more amino acid residues are added at the N- or C-terminus of, or within, a native artemin sequence. Artemin variants also include artemin polypeptides where a number of amino acid residues are deleted and optionally substituted by one or more amino acid residues. Artemin variants also may be covalently modified, for example by substitution with a moiety other than a naturally occurring amino acid or by modifying an amino acid residue to produce a non-naturally occurring amino acid.

“Percent amino acid sequence identity” with respect to the artemin sequence is defined herein as the percentage of amino acid residues in the candidate sequence that are identical with the residues in the artemin sequence, after aligning the sequences and introducing gaps, if necessary, to achieve the maximum percent sequence identity, and not considering any conservative substitutions as part of the sequence identity. None of N-terminal, C-terminal, or internal extensions, deletions or insertions into the candidate artemin sequence shall be construed as affecting sequence identity or homology. Methods and computer programs for the alignment are well known in the art. One such computer program is “ALIGN-2,” authored by Genentech, Inc., which has been filed with user documentation in the United States Copyright Office, Washington, D.C. 20559, where it is registered under U.S. Copyright Registration No. TXU510087.

A “chimeric artemin” molecule is a polypeptide comprising full-length artemin or one or more domains thereof fused or bonded to heterologous polypeptide. The chimeric artemin molecule will generally share at least one biological property in common with naturally occurring artemin. An example of a chimeric artemin molecule is one that is epitope tagged for purification purposes. Another chimeric artemin molecule is an artemin immunoadhesin.

The term “epitope-tagged” when used herein refers to a chimeric polypeptide comprising artemin fused to a “tag polypeptide”. The tag polypeptide has enough residues to provide an epitope against which an antibody can be made, yet is short enough such that it does not interfere with biological activity of the artemin. The tag polypeptide preferably is fairly unique so that the antibody against it does not substantially cross-react with other epitopes. Suitable tag polypeptides generally have at least six amino acid residues and usually between about 8-50 amino acid residues (preferably between about 9-30 residues). Preferred are poly-histidine sequences, which bind nickel, allowing isolation of the tagged protein by Ni-NTA chromatography as described (See, e.g., Lindsay et al. Neuron 17:571-574 (1996)).

Artemin “agonists” are molecules or compounds that have one or more of the biological properties of native sequence artemin. These may include, but are not limited to, small organic molecules, peptides, and agonist anti-artemin antibodies.

“Isolated artemin” means artemin that has been purified from an artemin source or has been prepared by recombinant or synthetic methods and purified. Purified artemin is substantially free of other polypeptides or peptides. “Substantially free” here means less than about 5%, preferably less than about 2%, more preferably less than about 1%, even more preferably less than about 0.5%, most preferably less than about 0.1% contamination with other source proteins.

“Essentially pure” protein means a composition comprising at least about 90% by weight of the protein, based on total weight of the composition, preferably at least about 95% by weight, more preferably at least about 90% by weight, even more preferably at least about 95% by weight. “Essentially homogeneous” protein means a composition comprising at least about 99% by weight of protein, based on total weight of the composition.

“Biological property” when used in conjunction with “artemin” or “isolated artemin” or an “agonist” of artemin, means having an effector or antigenic function or activity that is directly or indirectly caused or performed by native sequence artemin (whether in its native or denatured conformation). Effector functions include receptor binding (preferably with high affinity), and enhancement of survival, differentiation and/or proliferation of cells and the prevention of injury-induced changes in cells, especially neurons.

“Biologically active” when used in conjunction with “artemin” or “isolated artemin” or an agonist of artemin, means an artemin polypeptide that exhibits or shares an effector function of native sequence artemin. A principal effector function of artemin is its ability to stimulate the survival of neurons. Another principal effector function of artemin is its ability to prevent injury-induced changes in neurons. Without limitation, preferred biological activities include the ability to promote the development, proliferation, maintenance and/or regeneration of damaged neuronal cells in vitro or in vivo, including peripheral (sympathetic, parasympathetic, sensory, and. enteric) neurons, motorneurons, and central (brain and spinal cord) neurons. A particularly preferred biological activity is the ability to ameliorate (including treatment and prevention) a neuropathy, e.g. peripheral neuropathy or other neurodegenerative disease, or repair a damaged nerve cell. The damaged neurons may be sensory, sympathetic, parasympathetic; or enteric, e.g. dorsal root ganglia neurons, motorneurons, and central neurons, e.g. neurons from the spinal cord. The damage may be of any cause, including trauma, toxic agents, adverse side effects of other therapeutic agents, surgery, stroke, ischemia, infection, metabolic disease, nutritional deficiency, and various malignancies.

“Artemin receptor” is a molecule to which artemin binds and which mediates the biological properties of artemin.

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules that lack antigen specificity. Polypeptides of the latter kind are, for example, produced at low levels by the lymph system and at increased levels by myelomas.

“Native antibodies” and “native immunoglobulins” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intra-chain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light- chain variable domain is aligned with the variable domain of the heavy chain. Particular amino acid residues are believed to form an interface between the light- and heavy-chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework region (FR). The variable domains of native heavy and light chains each comprise four FRs (FR1, FR2, FR3 and FR4, respectively), largely adopting a sheet configuration, connected by three hypervariable regions, which form loops connecting, and in some cases forming part of, the sheet structure. The hypervariable regions in each chain are held-together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991), pages 647-669). The constant domains are not involved directly in binding an antibody to an antigen, but exhibit various effector functions, such as participation of the antibody in antibody-dependent cellular toxicity.

The term “hypervariable region” when used herein refers to the amino acid residues of an antibody which are responsible for antigen binding. The hypervariable region comprises amino acid residues from a “complementarity determining region” or ° “CDR” (i.e. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, J. Mol. Biol. 196:901-917 (1987)). “Framework” or “FR” residues are those variable domain residues other than the hypervariable region residues as herein defined.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Fv” is the minimum antibody fragment that contains a complete antigen-recognition and binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fᵥ comprising only three hypervariable regions specific for an antigen) has the ability to recognize and bind antigen, although at a lower affinity than the entire binding site.

The Fab fragrment also contains the constant domain of the light chain and the first constant domain (CH1) of the heavy chain. Fab′ fragments differ from Fab fragments by the addition of a few residues at the carboxyl terminus of the heavy chain CH1 domain including one or more cysteine(s) from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa ( ) and lambda ( ), based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy-chain constant domains that correspond to the different classes of immunoglobulins are called . . . , and, respectively. The subunit structures and three-dimensional configurations of different classes of immunoglobulins are well known.

The term “antibody” herein is used in the broadest sense and specifically covers human, non-human (e.g. murine) and humanized monoclonal antibodies (including full length monoclonal antibodies), polyclonal antibodies, multi-specific antibodies (e.g., bispecific antibodies), and antibody fragments so long as they exhibit the desired biological activity.

“Antibody fragments” comprise a portion of a full-length antibody, generally the antigen binding or variable domain thereof. Examples of antibody fragments include Fab, Fab′, F(ab′)₂, and Fv fragments; diabodies; linear antibodies; single-chain antibody molecules; and multi-specific antibodies formed from antibody fragments.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352:624628 (1991) and Marks et al., J. Mol. Biol. 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Humanized” forms of non-human (e.g., murine) antibodies are chimeric antibodies that contain minimal sequence derived from non-human immunoglobulin. For the most part, humanized antibodies are human immunoglobulins (recipient antibody) in which hypervariable region residues of the recipient are replaced by hypervariable region residues from a non-human species (donor antibody) such as mouse, rat, rabbit or non-human primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (11986); Reichmann et al., Nature 332:323-329 (1988); and Presta, Curr. Op. Struct. Biol. 2:593-596 (1992).

“Single-chain Fv” or “sFv” antibody fragments comprise the V_{H} and V_{L} domains of antibody, wherein these domains are present in a single polypeptide chain. Generally, the Fv polypeptide further comprises a polypeptide linker between the V_{H} and V_{L} domains which enables the sFv to form the desired structure for antigen binding. For a review of sFv see Pluckthun in The Pharmacology of Monoclonal Antibodies, vol. 113, Rosenburg and Moore eds. Springer-Verlag, N.Y., pp. 269-315 (1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA90:6444-6448 (1993).

The expression “linear antibodies” when used throughout this application refers to theantibodies described in Zapata et al. Protein Eng. 8(10):1057-1062 (1995). Briefly, these antibodies comprise a pair of tandem Fd segments (V_{H}-C_{H1}-V_{H}-C_{H1}) which form a pair of antigen binding regions. Linear antibodies can be bispecific or monospecific.

The term “epitope” is used to refer to binding sites for (monoclonal or polyclonal) antibodies on protein antigens.

By “agonist antibody” is meant an antibody that is an artemin agonist and thus possesses one or more of the biological properties of native sequence artemin.

The term “artemin immunoadhesin” is used interchangeably with the term, “artemin-immunoglobulin chimera”, and refers to a chimeric molecule that combines at least a portion of an artemin molecule (native or variant) with an immunoglobulin sequence. The immunoglobulin sequence preferably, but not necessarily, is an immunoglobulin constant domain. Immunoadhesins can possess many of the valuable chemical and biological properties of human antibodies. Since immunoadhesins can be constructed from a human protein sequence with a desired specificity linked to an appropriate human immunoglobulin hinge and constant domain (Fc) sequence, the binding specificity of interest can be achieved using entirely human components. Such immunoadhesins are minimally immunogenic to the patient, and are safe for chronic or repeated use.

Examples of homomultimeric immunoadhesins which have been described for therapeutic use include the CD4-IgG immunoadhesin for blocking the binding of HIV to cell-surface CD4. Data obtained from Phase I clinical trials, in which CD4-IgG was administered to pregnant women just before delivery, suggests that this immunoadhesin may be useful in the prevention of maternal-fetal transfer of HIV (Ashkenazi et al., Intern. Rev. Immunol. 10:219-227 (1993)). An immunoadhesin which binds tumor necrosis factor (TNF) has also been developed. TNF is a proinflammatory cytokine which has been shown to be a major mediator of septic shock. Based on a mouse model of septic shock, a TNF receptor immunoadhesin has shown promise as a candidate for clinical use in treating septic shock (Ashkenazi, A. et al., (1991) PNAS USA 88:10535-10539). ENBREL® (etanercept), an immunoadhesin comprising a TNF receptor sequence fused to an IgG Fc region, was approved by the U.S. Food and Drug Administration (FDA), on Nov. 2, 1998, for the treatment of rheumatoid arthritis. The new expanded use of ENBREL® in the treatment of rheumatoid arthritis was approved by FDA on Jun. 6, 2000. For recent information on TNF blockers, including EMBREL®, see Lovell et al., N. Engl. J. Med 342: 763-169 (2000), and accompanying editorial on p810-811; and Weinblatt et al., N. Engl. J. Med. 340: 253-259 (1999); reviewed in Maini and Taylor, Annu. Rev. Med. 51: 207-229 (2000).

If the two arms of the immunoadhesin structure have different specificities, the immunoadhesin is called a “bispecific immunoadhesin” by analogy to bispecific antibodies. Dietsch et al., J. Immunal. Methods 162:123 (1993) describe such a bispecific immunoadhesin combining the extracellular domains of the adhesion molecules, E-selectin and P-selectin, each of which selectins is expressed in a different cell type in nature. Binding studies indicated that the bispecific immunoglobulin fusion protein so formed had an enhanced ability to bind to a myeloid cell line compared to the monospecific immunoadhesins from which it was derived.

The term “heteroadhesin” is used interchangeably with the expression “chimeric heteromultimer adhesin” and refers to a complex of chimeric molecules (amino acid sequences) in which each chimeric molecule combines a biologically active portion, such as the extracellular domain of each of the heteromultimeric receptor monomers, with a multimerization domain. The “multimerization domain” promotes stable interaction of the chimeric molecules within the heteromultimer complex. The multimerization domains may interact via an immunoglobulin sequence, leucine zipper, a hydrophobic region, a hydrophilic region, or a free thiol which forms an intermolecular disulfide bond between the chimeric molecules of the chimeric heteromultimer. The multimerization domain may comprise an immunoglobulin constant region. In addition a multimerization region may be engineered such that steric interactions not only promote stable interaction, but further promote the formation of heterodimers over homodimers from a mixture of monomers. “Protuberances” are constructed by replacing small amino acid side chains from the interface of the first polypeptide with larger side chains (e.g. tyrosine or tryptophan). Compensatory “cavities” of identical or similar size to the protuberances are optionally created on the interface of the second polypeptide by replacing large amino acid side chains with smaller ones (e.g. alanine or threonine). The immunoglobulin sequence preferably, but not necessarily, is an immunoglobulin constant domain. The immunoglobulin moiety in the chimeras of the present invention may be obtained from IgG₁, IgG₂, IgG₃ or IgG₄ subtypes, IgA, IgE, IgD or IgM, but preferably IgG₁ or IgG₃.

As used herein, “treatment” is an approach for obtaining beneficial or desired clinical results. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. “Treatment” is an intervention performed with the intention of preventing the development or altering the pathology of a disorder. Accordingly, “treatment” refers to both therapeutic treatment and prophylactic or preventative measures. Those in need of treatment include those already with the disorder as well as those in which the disorder is to be prevented. Specifically, the treatment may directly prevent, slow down or otherwise decrease the pathology of cellular degeneration of damage, such as the pathology of nerve cells, or may render the cells, e.g. neurons more susceptible to treatment by other therapeutic agents. In a preferred embodiment, the treatment reduces or slows down the decline and/or stimulates the restoration of the function of target neurons.

The “pathology” of a (chronic) neurodegenerative disease or acute nervous system injury includes all phenomena that affect the well being of the patient including, without limitation, neuronal disfunction, degeneration, injury and/or death.

The terms “neurodegenerative disease” and “neurodegenerative disorder” are used in the broadest sense to include all disorders the pathology of which involves neuronal degeneration and/or disfunction, including, without limitation, peripheral neuropathies; motorneuron disorders, such as amyotrophic lateral sclerosis (ALS), Lou Gehrig's disease, Bell's palsy, and various conditions involving spinal muscular atrophy or paralysis; and other human neurodegenerative diseases, such as Alzheimer's disease, Parkinson's disease, epilepsy, multiple sclerosis, Huntington's chorea, Down's Syndrome, nerve deafness, and Nebuere's disease.

“Peripheral neuropathy” is a neurodegenerative disorder that affects the peripheral nerves, most often manifested as one or a combination of motor, sensory, sensorimotor, or autonomic dysfunction. Peripheral neuropathies may, for example, be genetically acquired, can result from a systemic disease, or can be induced by a toxic agent, such as a neurotoxic drug, e.g. an antineoplastic agent, or industrial or environmental pollutant. “Peripheral sensory neuropathy” is characterized by the degeneration of peripheral sensory neurons, which may be idiopathic, may occur, for example, as a consequence of diabetes (diabetic neuropathy), cytostatic drug therapy in cancer (e.g. treatment with chemotherapeutic agents such as vincristine, cisplatin, methotrexate, 3′-azido-3′-deoxythymidine, or taxanes, e.g. paclitaxel [TAXOL®, Bristol-Myers Squibb Oncology, Princeton, N.J.] and doxetaxel [TAXOTERE®, Rhöne-Poulenc Rorer, Antony, France]), alcoholism, acquired immunodeficiency syndrom (AIDS), or genetic predisposition. Genetically acquired peripheral neuropathies include, for example, Refsum's disease, Krabbe's disease, Metachromatic leukodystrophy, Fabry's disease, Dejerine-Sottas syndrome, Abetalipoproteinemia, and Charcot-Marie-Tooth (CMT) Disease (also known as Proneal Muscular Atrophy or Hereditary Motor Sensory Neuropathy (HMSN)). Most types of peripheral neuropathy develop slowly, over the course of several months or years. In clinical practice such neuropathies are called chronic. Sometimes a peripheral neuropathy develops rapidly, over the course of a few days, and is referred to as acute. Peripheral neuropathy usually affects sensory and motor nerves together so as to cause a mixed sensory and motor neuropathy, but pure sensory and pure motor neuropathy are also known.

The term “toxic agent”, as used in the context of the present invention, is meant to refer to a substance that, through its chemical action, injures, impairs, or inhibits the activity of a component of the nervous system. The long list of toxic agents (also referred to as “neurotoxic agents”) includes, without limitation, chemotherapeutic agents, such as those fisted above, alcohol, metals, industrial toxins, contaminants of food and medicines, etc.

“Mammal” for purpose of treatment refers to any animal classified as a mammal, including humans, domestic and farm animals, and zoo, sport or pet animals, such as dogs, horses, sheep, cats, cows, etc. Preferably, the mammal is human.

In the context of the present invention the expressions “cell”, “cell line”, and “cell culture” are used interchangeably, and all such designations include progeny. Thus, the words “transformants” and “transformed (host) cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

An “exogenous” element is defined herein to mean nucleic acid sequence that is foreign to the cell, or homologous to the cell but in a position within the host cell nucleic acid in which the element is ordinarily not found.

B. Methods for Carrying Out the Invention

1. Identification of Artemin Variants

In addition to the full-length native sequence artemin polypeptides described herein, it is contemplated that artemin variants can be identified, prepared and used in the present invention. Artemin variants can be prepared by introducing appropriate nucleotide changes into the artemin DNA, and/or by synthesis of the desired artemin polypeptide. Those skilled in the art will appreciate that amino acid changes may alter post-translational processes of the artemin, such as changing the number or position of glycosylation sites. The methods of production of artemin variants are preferably the same as for native sequence artemin as described in detail below, the only difference being the substitution of the nucleic acid encoding the artemin variant for the nucleic acid encoding native sequence artemin.

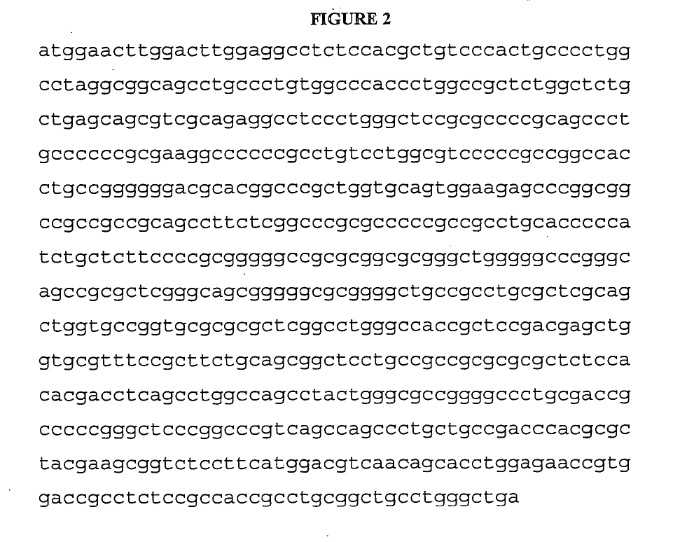

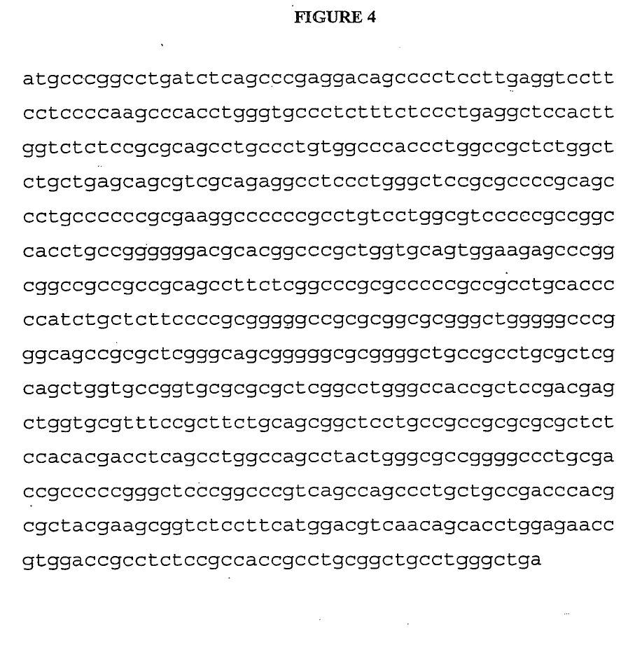

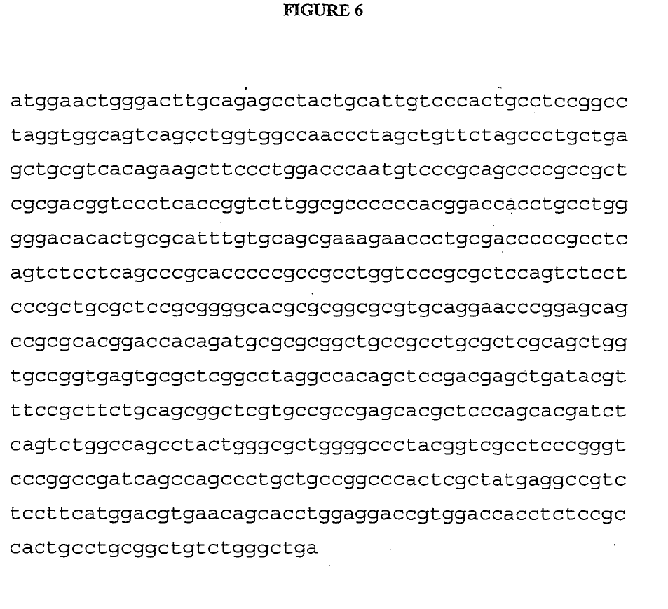

Nucleic acid molecules that encode artemin are used in the methods of the present invention. cDNAs encoding two full-length variants of human artemin are provided in FIGS. 2 and 4 (SEQ ID NOS: 2 and 4), and the corresponding deduced amino acid sequences are provided in FIGS. 3 and 5 (SEQ ID NOS: 3 and 5). A cDNA encoding mouse artemin is provided in FIG. 6 (SEQ ID NO: 6) and the corresponding deduced amino acid sequence is provided in FIG. 7 (SEQ ID NO: 7). The polynucleotides used in the present invention can be obtained using standard techniques well known to those skilled in the art such as, for example, hybridization screening and PCR methodology.

Any nucleotide sequence which encodes the amino acid sequence of artemin can be used to generate recombinant molecules which direct the expression of artemin. Additionally, the methods of the present invention may also utilize a fusion polynucleotide between an artemin coding sequence and a second coding sequence for a heterologous protein.

In order to clone full length homologous cDNA sequences from any species encoding the entire artemin cDNA or to clone family members or variant forms such as allelic variants, labeled DNA probes made from fragments corresponding to any part of the cDNA sequences disclosed herein may be used to screen a cDNA library derived from a cell or tissue type believed to express artemin. More specifically, oligonucleotides corresponding to either the 5′ or 3′ terminus of the coding sequence may be used to obtain longer nucleotide sequences.

It may be necessary to screen multiple cDNA libraries from different tissues to obtain a full-length cDNA. In the event that it is difficult to identify cDNA clones encoding the complete 5′ terminal coding region, an often encountered situation in cDNA cloning, the RACE (Rapid Amplification of cDNA Ends) technique may be used. RACE is a proven PCR-based strategy for amplifying the 5′ end of incomplete cDNAs. 5′-RACE-Ready RNA synthesized from human placenta containing a unique anchor sequence is commercially available (Clontech). To obtain the 5′ end of the cDNA, PCR is carried out on 5′-RACE-Ready cDNA using the provided anchor primer and the 3′ primer. A secondary PCR is then carried out using the anchored primer and a nested 3′ primer according to the manufacturer's instructions. Once obtained, the full length cDNA sequence may be translated into amino acid sequence and examined for certain landmarks such as a continuous open reading frame flanked by translation initiation and termination sites, a potential signal sequence and finally overall structural similarity to the artemin sequences disclosed herein.

Alternatively, a labeled probe may be used to screen a genomic library derived from any organism of interest using appropriate stringent conditions as described infra.

Isolation of an artemin coding sequence or a homologous sequence may be carried out by the polymerase chain reactions (PCR) using two degenerate oligonucleotide primer pools designed on the basis of the artemin coding sequences disclosed herein. The template for the reaction may be cDNA obtained by reverse transcription (RT) of mRNA prepared from, for example, human or non-human cell lines or tissues known or suspected to express an artemin gene allele.

The PCR product may be subcloned and sequenced to ensure that the amplified sequences represent the sequences of an artemin coding sequence. The PCR fragment may then be used to isolate a full-length cDNA clone by a variety of methods. For example, the amplified fragment may be labeled and used to screen a bacteriophage cDNA library. Alternatively, the labeled fragment may be used to isolate genomic clones via the screening of a genomic library.

PCR technology may also be utilized to isolate full-length cONA sequences. For example, RNA may be isolated, following standard procedures, from an appropriate cellular or tissue source. An RT reaction may be performed on the RNA using an oligonucleotide primer specific for the most 5′ end of the amplified fragment for the priming of first strand synthesis. The resulting RNA/DNA hybrid may then be “tailed” with guanines using a standard terminal transferase reaction, the hybrid may be digested with RNAase H, and second strand synthesis may then be primed with a poly-C primer. Thus, cDNA sequences upstream of the amplified fragment may easily be isolated.

A cDNA clone of a mutant or allelic variant of the artemin gene may be isolated, for example, by using PCR. In this case, the first cDNA strand may be synthesized by hybridizing an oligo-dT oiigonucleotide to mRNA isolated from tissue known or suspected to be expressed in an individual putatively carrying the mutant artemin allele, and by extending the new strand with reverse transcriptase. The second strand of the cDNA is then synthesized using an oligonucleotide that hybridizes specifically to the 5′ end of the normal gene. Using these two primers, the product is then amplified via PCR, cloned into a suitable vector, and subjected to DNA sequence analysis through methods well known to those of skill in the art. By comparing the DNA sequence of the mutant artemin allele to that of the normal artemin allele, the mutation(s) responsible for the loss or alteration of function of the mutant artemin gene product can be ascertained.

Alternatively, a genomic library can be constructed using DNA obtained from an individual suspected of or known to carry a mutant artemin allele, or a cDNA library can be constructed using RNA from a tissue known, or suspected, to express a mutant artemin allele. An unimpaired artemin gene or any suitable fragment thereof may then be labeled and used as a probe to identify the corresponding mutant artemin allele in such libraries. Clones containing the mutant artemin gene sequences may then be purified and subjected to sequence analysis according to methods well known to those of skill in the art.

Additionally, an expression library can be constructed utilizing cDNA synthesized from, for example, RNA isolated from a tissue known, or suspected, to express a mutant artemin allele in an individual suspected of or known to carry such a mutant allele. In this manner, gene products made by the putatively mutant tissue may be expressed and screened using standard antibody screening techniques in conjunction with antibodies raised against the normal artemin gene product, as described, below. As used herein, the terms nucleic acid, polynucleotide and nucleotide are interchangeable and refer to any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages.

The terms nucleic acid, polynucleotide and nucleotide also specifically include nucleic acids composed of bases other than the five biologically occurring bases (adenine, guanine, thymine, cytosine and uracil). For example, a polynucleotide of the invention might contain at least one modified base moiety which is selected from the group including but not limited to 5-fluorouracil, 5-bromouracil, 5-chlorouracil, 5-iodouracil, hypoxanthine, xantine, 4-acetylcytosine, 5(carboxyhydroxylmethyl) uracil, 5carboxymethylaminomethyl-2-thiouridine, 5carboxymethylaminomethyl-uracil, dihydrouracil, beta-D-galactosylqueosine, inosine, N6-isopentenyladenine, 1-methylguanine, 1-methylinosine, 2,2-dimethylguanine, 2-methyladenine, 2methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl2-thiouracil, 3-(3amino-3-N-2carboxypropyl) uracil, (acp3)w, and 2,6-diaminopurine.

Furthermore, a polynucleotide used in the invention may comprise at least one modified sugar moiety selected from the group including but not limited to arabinose, 2-fluoroarabinose, xylulose, and hexose.

It is not intended that the methods of the present invention be limited by the source of the polynucleotide. The polynucleotide can be from a human or non-human mammal, derived from any recombinant source, synthesized in vitro or by chemical synthesis. The nucleotide may be DNA or RNA and may exist in a double-stranded, single-stranded or partially double-stranded form.

Nucleic acids useful in the present invention include, by way of example and not limitation, oligonucleotides such as antisense DNAs andlor RNAs; ribozymes; DNA for gene therapy; DNA andlor RNA chimeras; various structural forms of DNA including single-stranded DNA, double-stranded DNA, supercoiled DNA and/or triple-helix DNA; Z-DNA; and the like. The nucleic acids may be prepared by any conventional means typically used to prepare nucleic acids in large quantity. For example, DNAs and RNAs may be chemically synthesized using commercially available reagents and synthesizers by methods that are well-known in the art (see, e.g., Gait, 1985, Oligonucleotide Synthesis: A Practical Approach, IRL Press, Oxford, England). RNAs may be produce in high yield via in vitro transcription using plasmids such as SP65 (Promega Corporation, Madison, Wis.).

Any mRNA transcript encoded by artemin nucleic acid sequences may be used in the methods of the present invention, including in particular, mRNA transcripts resulting from alternative splicing or processing of mRNA precursors.

In some circumstances, as where increased nuclease stability is desired, nucleic acids having modified internucleoside linkages may be preferred. Nucleic acids containing modified internucleoside linkages may also be synthesized using reagents and methods that are well known in the art. For example, methods for synthesizing nucleic acids containing phosphonate phosphorothioate, phosphorodithioate, phosphoramidate methoxyethyl phosphoramidate, formacetal, thioformacetal, diisopropylsilyl, acetamidate, carbamate, dimethylene-sulfide (—CH₂—S—CH₂), dimethylene-sulfoxide (—CH₂—SO—CH₂), dimethylene-sulfone (—CH₂—SO₂—CH₂), 2′-O-alkyl, and 2′-deoxy-2′-fluoro phosphorothioate internucleoside linkages are well known in the art (see Uhlmann et al., 1990, Chem. Rev. 90:543-584; Schneider et al., 1990, Tetrahedron Lett. 31:335 and references cited therein).

In some embodiments of the present invention, the nucleotide used is an anomeric nucleotide. An anomeric nucleotide forms specific double-stranded hybrids with complementary RNA in which, contrary to the usual units, the strands run parallel to each other (Gautier et al., 1987, Nucl. Acids Res. 15:6625-6641). The nucleotide is a 2-O-methylribonucleotide (Inoue et al., 1987, Nucl. Acids Res. 15:6131-6148), or a chimeric RNA-DNA analogue (Inoue et al., 1987, FEBS Lett.215:327-330).

The nucleic acids may be purified by any suitable means, as are well known in the art. For example, the nucleic acids can be purified by reverse phase or ion exchange HPLC, size exclusion chromatography or gel electrophoresis. Of course, the skilled artisan will recognize that the method of purification will depend in part on the size of the DNA to be purified.

Isolated or purified polynucleotides having at least 10 nucleotides (i.e., a hybridizable portion) of an artemin coding sequence or its complement may also be used in the methods of the present invention. In other embodiments, the polynucleotides contain at least 25 (continuous) nucleotides, 50 nucleotides, 1 00 nucleotides, 150 nucleotides, or 200 nucleotides of an artemin coding sequence, or a full-length artemin coding sequence. Nucleic acids can be single or double stranded. Additionally, the invention relates to polynucleotides that selectively hybridize to a complement of the foregoing coding sequences. In preferred embodiments, the polynucleotides contain at least 10, 25, 50, 100, 150 or 200 nucleotides or the entire length of an artemin coding sequence.

Nucleotide sequences that encode a mutant of artemin, peptide fragments of artemin, truncated forms of artemin, and artemin fusion proteins may also be useful in the methods of the present invention. Nucleotides encoding fusion proteins may include, but are not limited to, full length artemin sequences, truncated forms of artemin, or nucleotides encoding peptide fragments of artemin fused to an unrelated protein or peptide, such as far example, a domain fused to an Ig Fc domain which increases the stability and half life of the resulting fusion protein (e.g., artemin-Ig) in the bloodstream; or an enzyme such as a fluorescent protein or a luminescent protein which can be used as a marker.

Furthermore, artemin polynucleotide variants that have been generated, at least in part, by some form of directed evolution, e.g., gene shuffling andlor recursive sequence recombination, described in U.S. Pat. Nos. 5,605,793 and 5,837,458, incorporated by reference herein in their entirety, may be used in the methods of the present invention. For example, using such techniques one can use an artemin encoding sequence, or a plurality of artemin encoding sequences, as the starting point for the generation of novel sequences encoding functionally and/or structurally similar proteins with altered functional and/or structural characteristics.

Highly related gene homologs of the artemin encoding polynucleotide sequences described above may also be useful in the present invention. Highly related gene homologs are polynucleotides encoding proteins that have at least about 60% amino acid sequence identity with the amino acid sequence of a naturally occurring artemin such as the mature human artemin of FIG. 1 (SEQ 10 NO: 1), preferably at least about 65%, 70%, 75%, 80%, with increasing preference of at least about 85% to at least about 99% amino acid sequence identity, in 1% increments. Highly related homologs can encode proteins sharing functional activities with artemin.

The methods of the present invention also benefit by the use of (a) DNA vectors that contain any of the foregoing artemin coding sequences andlor their complements (i.e., antisense); (b) DNA expression vectors that contain any of the foregoing artemin coding sequences operatively associated with a regulatory element that directs the expression of the coding sequences; (c) genetically engineered host cells that contain any of the foregoing artemin coding sequences operatively associated with a regulatory element that directs the expression of the coding sequences in the host cell; and (d) genetically engineered host cells that express an endogenous artemin gene under the control of an exogenously introduced regulatory element (i.e., gene activation).

Variations in native sequence artemin or in various domains of the artemin described herein, can be made, for example, using any of the techniques and guidelines for conservative and nonconservative mutations set forth, for instance, in U.S. Pat. No. 5,364,934. Variations may be a substitution, deletion or insertion of one or more codons encoding artemin that results in a change in the amino acid sequence of the artemin as compared with native sequence artemin. Optionally the variation is by substitution of at least one amino acid with any other amino acid in one or more of the domains of the artemin. Guidance in determining which amino acid residue may be inserted, substituted or deleted without adversely affecting the desired activity may be found by comparing the sequence of artemin with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence.

Artemin polypeptide fragments are also useful in the methods of the present invention. Such fragments may be truncated at the N-terminus or C-terminus, or may lack internal residues, for example, when compared with a full-length native protein. Certain fragments lack amino acid residues that are not essential for a desired biological activity of the artemin polypeptide.

Artemin fragments may be prepared by any of a number of conventional techniques. Desired peptide fragments may be chemically synthesized. An alternative approach involves generating artemin fragments by enzymatic digestion, e.g., by treating the protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding a desired polypeptide fragment, by polymerase chain reaction (PCR). Oligonucleotides that define the desired termini of the DNA fragment are employed at the 5′ and 3′ primers in the PCR. Preferably, artemin polypeptide fragments share at least one biological and/or immunological activity with the native artemin polypeptide shown in FIG. 1 (SEQ ID NO: 1).

In particular embodiments, conservative substitutions of interest are shown in Table 1 under the heading of preferred substitutions. If such substitutions result in a change in biological activity, then more substantial changes, denominated exemplary substitutions in Table 1, or as further described below in reference to amino acid classes, are introduced and the products screened.

TABLE 1

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; | leu |
| | phe; norleucine | |
| Leu (L) | norleucine; ile; val | ile |
| | met; ala; phe | |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe | leu |
| | ala; norleucine | |

Substantial modifications in function or immunological identity of the artemin polypeptide are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic: trp, tyr, phe.

Non-conservative substitutions will entail exchanging a member of one of these classes for another class. Such substituted residues also may be introduced into the conservative substitution sites or, more preferably, into the remaining (non-conserved) sites.

The variations can be made using methods known in the art such as oligonucleotide-mediated (site-directed) mutagenesis, alanine scanning, and PCR mutagenesis. Site-directed mutagenesis [Carter et al., Nucl. Acids Res., 13:4331 (1986); Zoller et al., Nucl. Acids Res., 10:6487 (1987)], cassette mutagenesis [Wells et al., Gene, 34:315 (1985)], restriction selection mutagenesis [Wells et al., Philos. Trans. R. Soc. London SerA, 317:415 (1986)] or other known techniques can be performed on cloned DNA to produce the artemin variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant [Cunningham and Wells, Science, 244: 1081-1085 (1989)]. Alanine is also typically preferred because it is the most common amino acid. Further, it is frequently found in both buried and exposed positions [Creighton, The Proteins,(W. H. Freeman & Co., N.Y.); Chothia, J. Mol. Biol., 150:1 (1976)]. If alanine substitution does not yield adequate amounts of variant, an isoteric amino acid can be used.

2. Production of Artemin and Artemin Variants

Techniques suitable for the production of artemin and artemin variants are well known in the art. Because the preferred techniques are the same for artemin and artemin variants, the techniques described below apply to artemin variants as well as to native sequence artemin.

The preferred methods of production include isolating artemin from an endogenous source of the polypeptide, peptide synthesis (using a peptide synthesizer) and recombinant techniques (or any combination of these techniques). Methods of preparing artemin using a recombinant technique are described in Baloh et al., Neuron 21:1291-1302 (1998) and in WO 00118799. Other recombinant techniques are described below.

Most of the discussion below pertains to recombinant production of artemin by culturing cells transformed with a vector containing artemin nucleic acid and recovering the polypeptide from the cell culture. It is further envisioned that the artemin of this invention may be produced by homologous recombination, as provided for in WO 91/06667, published 16 May 1991.

Briefly, this method involves transforming primary human cells containing an artemin-encoding gene with a construct (i.e., vector) comprising an amplifiable gene (such as dihydrofolate reductase (DHFR) or others discussed below) and at least one flanking region of a length of at least about 150 bp that is homologous with a DNA sequence at the locus of the coding region of the artemin gene to provide amplification of the artemin gene. The amplifiable gene must be at a site that does not interfere with expression of the artemin gene. The transformation is conducted such that the construct becomes homologously integrated into the genome of the primary cells to define an amplifiable region.

Primary cells comprising the construct are then selected for by means of the amplifiable gene or other marker present in the construct. The presence of the marker gene establishes the presence and integration of the construct into the host genome. No further selection of the primary cells need be made, since selection will be made in the second host. If desired, the occurrence of the homologous recombination event can be determined by employing PCR and either sequencing the resulting amplified DNA sequences or determining the appropriate length of the PCR fragment when DNA from correct homologous integrants is present and expanding only those cells containing such fragments. Also if desired, the selected cells may be amplified at this point by stressing the cells with the appropriate amplifying agent (such as methotrexate if the amplifiable gene is DHFR), so that multiple copies of the target gene are obtained. Preferably, however, the amplification step is not conducted until after the second transformation described below.

After the selection step, DNA portions of the genome, sufficiently large to include the entire amplifiable region, are isolated from the selected primary cells. Secondary mammalian expression host cells are then transformed with these genomic DNA portions and cloned, and clones are selected that contain the amplifiable region. The amplifiable region is then amplified by means of an amplifying agent if not already amplified in the primary cells. Finally, the secondary expression host cells now comprising multiple copies of the amplifiable region containing artemin are grown so as to express the gene and produce the protein.

The DNA encoding artemin may be obtained from any cDNA library prepared from tissue believed to possess the artemin mRNA and to express it at a detectable level. Accordingly, artemin DNA can be conveniently obtained from a cDNA library prepared, for example, from multiple human tissues. The artemin-encoding gene may also be obtained from a genomic library or by oligonucleotide synthesis.

Libraries are screened with probes (such as antibodies to artemin or oligonucleotides of about 20-80 bases) designed to identify the gene of interest or the protein encoded by it. Screening the cDNA or genomic library with the selected probe may be conducted using standard procedures as described in chapters 10.12 of Sambrook et al., Molecular Cloning: A Laboratory Manual (New York: Cold Spring Harbor Laboratory Press, 1989). An alternative means to isolate the gene encoding artemin is to use PCR methodology as described in section 14 of Sambrook et al., supra.

A preferred method of isolating artemin cDNA is to use carefully selected oligonucleotide sequences to screen cDNA libraries from various human tissues. The oligonucleotide sequences selected as probes should be of sufficient length and sufficiently unambiguous that false positives are minimized. Preferred sequences are obtained from the naturally occurring artemin disclosed herein.

The oligonucleotide must be labeled such that it can be detected upon hybridization to DNA in the library being screened. The preferred method of labeling is to use ³²P-labeled ATP with polynucleotide kinase, as is well known in the art, to radiolabel the oligonucleotide. However, other methods may be used to label the oligonucleotide, including, but not limited to, biotinylation or enzyme labeling.

The nucleic acid (e.g., cDNA or genomic DNA) encoding artemin is inserted into a replicable vector for further cloning (amplification of the DNA) or for expression. Many vectors are available. The vector components generally include, but are not limited to, one or more of the following: a signal sequence, an origin of replication, one or more marker genes, an enhancer element, a promoter, and a transcription termination sequence.

The artemin of this invention may be produced recombinantly not only directly, but also as a fusion polypeptide with a heterologous polypeptide, which is preferably a signal sequence or other polypeptide having a specific cleavage site at the N-terminus of the mature protein or polypeptide. In general, the signal sequence may be a component of the vector, or it may be a part of the artemin DNA that is inserted into the vector. The heterologous signal sequence selected preferably is one that is recognized and processed (i.e., cleaved by a signal peptidase) by the host cell. For prokaryotic host cells that do not recognize and process the native artemin signal sequence, the signal sequence is substituted by a prokaryotic signal sequence selected, for example, from the group of the alkaline phosphatase, penicillinase, Ipp, or heat-stable enterotoxin II leaders. For yeast secretion the native signal sequence may be substituted by, e.g., the yeast invertase leader, factor leader (including Saccharomyces and Kluyveromyces factor leaders, the latter described in U.S. Pat. No. 5,010,182 issued 23 Apr. 1991), or acid phosphatase leader, the C. albicans glucoamylase leader (EP 362,179 published 4 Apr. 1990), or the signal described in WO 90/13646 published 15 Nov. 1990. In mammalian cell expression the native signal sequence (e.g. the artemin presequence that normally directs secretion of artemin from human cells in vivo) is satisfactory, although other mammalian signal sequences may be suitable, such as signal sequences from other animal artemins, and signal sequences from secreted polypeptides of the same or related species, as well as viral secretory leaders, for example, the herpes simplex gO signal.

The DNA for such precursor region is ligated in reading frame to DNA encoding the mature artemin or a soluble variant thereof.

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gramznegative bacteria, the 2 plasmid origin is suitable for yeast, and various viral origins (SV40, polyoma, adenovirus, VSV. or BPV) are useful for cloning vectors in mammalian cells.

Generally, the origin of replication component is not needed for mammalian expression vectors (the SV40 origin may typically be used only because it contains the early promoter).

Most expression vectors are “shuttle” vectors, i.e., they are capable of replication in at least one class of organisms but can be transfected into another organism for expression. For example, a vector is cloned in E. coli and then the same vector is transfected into yeast or mammalian cells for expression even though it is not capable of replicating independently of the host cell chromosome.

DNA may also be amplified by insertion into the host genome. This is readily accomplished using Bacillus species as hosts, for example, by including in the vector a DNA sequence that is complementary to a sequence found in Bacillus genomic DNA. Transfection of Bacillus with this vector results in homologous recombination with the genome and insertion of artemin DNA. However, the recovery of genomic DNA encoding artemin is more complex than that of an exogenously replicated vector because restriction enzyme digestion is required to excise the artemin DNA.

Expression and cloning vectors should contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up the artemin nucleic acid, such as DHFR or thymidine kinase. The mammalian cell transformants are placed under selection pressure that only the transformants are uniquely adapted to survive by virtue of having taken up the marker. Selection pressure is imposed by culturing the transformants under conditions in which the concentration of selection agent in the medium is successively changed, thereby leading to amplification of both the selection gene and the DNA that encodes artemin. Amplification is the process by which genes in greater demand for the production of a protein critical for growth are reiterated in tandem within the chromosomes of successive generations of recombinant cells. Increased quantities of artemin are synthesized from the amplified DNA. Other examples of amplifiable genes include metallothionein-I and -II, preferably primate metallothionein genes, adenosine deaminase, ornithine decarboxylase, etc. A preferred vector system is provided in U.S. Pat. No. 5,561,053.

For example, cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity, prepared and propagated as described by Urlaub et al., Proc. Natl. Acad. Sci. USA, 77:4216 (1980). The transformed cells are then exposed to increased levels of methotrexate. This leads to the synthesis of multiple copies of the DHFR gene, and, concomitantly, multiple copies of other DNA comprising the expression vectors, such as the DNA encoding artemin. This amplification technique can be used with any otherwise suitable host, e.g., ATCC No. CCL61 CHO-K1, notwithstanding the presence of endogenous DHFR if, for example, a mutant DHFR gene that is highly resistant to Mtx is employed (EP 117,060).

Alternatively, host cells (particularly wild-type hosts that contain endogenous DHFR) transformed or co-transformed with DNA sequences encoding artemin, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

A suitable selection gene for use in yeast is the trp1 gene present in the yeast plasmid YRp7 (Stinchcomb et al., Nature, 282:39 (1979)1. The trp1 gene provides a selection marker for a mutant strain of yeast lacking the ability to grow in tryptophan, for example, ATCC No. 44076 or PEP4-1. Jones, Genetics, 85:12 (1977). The presence of the trp1 lesion in the yeast host cell genome then provides an effective environment for detecting transformation by growth in the absence of tryptophan. Similarly, Leu2-deficient yeast strains (ATCC 20,622 or 38,626) are complemented by known plasmids bearing the Leu2 gene.

In addition, vectors derived from the 1.6 m circular plasmid pKD1 can be used for transformation of Kluyveromyces yeasts. Bianchi et al., Curr. Genet., 12:185 (1987). More recently, an expression system for large-scale production of recombinant calf chymosin was reported for K. lactis. Van den Berg, Bio/Technology, 8:135 (1990). Stable multi-copy expression vectors for secretion of mature recombinant human serum albumin by industrial strains of Kluyveromyces have also been disclosed. Fleer et al., Bio/Technology, 9:968-975 (1991).

Expression and cloning vectors usually contain a promoter that is recognized by the host organism and is operably linked to the artemin nucleic acid. Promoters are untranslated sequences located upstream (5′) to the start codon of a structural gene (generally within about 100 to 1000 bp) that control the transcription and translation of particular nucleic acid sequence, such as the artemin nucleic acid sequence, to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. At this time a large number of promoters recognized by a variety of potential host cells are well known. These promoters are operably linked to artemin-encoding DNA by removing the promoter from the source DNA by restriction enzyme digestion and inserting the isolated promoter sequence into the vector. Both the native artemin promoter sequence and many heterologous promoters may be used to direct amplification andlor expression of the artemin DNA. However, heterologous promoters are preferred, as they generally permit greater transcription and higher yields of artemin as compared to the native artemin promoter.

Promoters suitable for use with prokaryotic hosts include the lactamase and lactose promoter systems (Chang et al., Nature, 275:615 (1978); Goeddel et al., Nature, 281:544 (1979)), alkaline phosphatase, a tryptophan (trp) promoter system (Goeddel, Nucleic Acids Res., 8:4057 (1980); EP 36,776), and hybrid promoters such as the tac promoter. deBoer et al., Proc. Natl Acad. Sci. USA, 80:21.25 (1983). However, other known bacterial promoters are suitable. Their nucleotide sequences have been published, thereby enabling a skilled worker operably to ligate them to DNA encoding artemin (Siebenlist et al., Cell, 20:269 (1980)) using linkers or adaptors to supply any required restriction sites. Promoters for use in bacterial systems also will contain a Shine-Delgarno (S.D.) sequence operably linked to the DNA encoding artemin.

Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CXCAAT region where X may be any nucleotide. At the 3′ end of most eukaryotic genes is an AATAAA sequence that may be the signal for addition of the poly-A tail to the 3′ end of the coding sequence. All of these sequences are suitably inserted into eukaryotic expression vectors.

Examples of suitable promoting sequences for use with yeast hosts include the promoters for 3-phosphoglycerate kinase (Hitzeman et al., J. Biol. Chem., 255:2073 (1980)) or other glycolytic enzymes (Hess et al., J. Adv. Enzyme Reg., 7:149 (1968); Holland, Biochemistry, 17:4900 (1978)), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657. Yeast enhancers also are advantageously used with yeast promoters.

Artemin transcription from vectors in mammalian host cells is controlled, for example, by promoters obtained from the genomes of viruses such as polyoma virus, fowipax virus (UK 2,211,504 published 5 Jul. 1989), adenovirus (such as Adenovinus 2), bovine papilloma virus, avian sarcoma virus, cytomegalovirus, a retrovirus, hepatitis-B virus and most preferably Simian Virus 40(SV40), from heterologous mammalian promoters, e.g., the actin promoter or an immunoglobulin promoter, from heat-shock promoters, and from the promoter normally associated with the artemin sequence, provided such promoters are compatible with the host cell systems.

The early and late promoters of the SV40 virus are conveniently obtained as an SV40 restriction fragment that also contains the SV40 viral origin of replication. Fiers et al., Nature, 273:113 (1978); Mulligan et al., Science, 209:1422-1427 (1980); Pavlakis et al., Proc. Natl. Acad. Sci. USA, 78:7398-7402 (1981). The immediate early promoter of the human cytomegalovirus is conveniently obtained as a HindlIl E restriction fragment. Greenaway et al., Gene, 18:355-360 (1982). A system for expressing DNA in mammalian hosts using the bovine papilloma virus as a vector is disclosed in U.S. Pat. No. 4,419,446. A modification of this system is described in U.S. Pat. No. 4,601,978. See also Gray et al., Nature, 295:503-508 (1982) on expressing cDNA encoding immune interferon in monkey cells; Reyes et al., Nature, 297:598-601 (1982) on expression of human interferon cONA in mouse cells under the control of a thymidine kinase promoter from herpes simplex virus; Canaani et al., Proc. latl. Acad. Sci. USA, 79:5166-5170 (1982) on expression of the human interferon 1 gene in cultured mouse and rabbit cells; and Gorman et al., Proc. Natl. Acad. Sci. USA, 79:6777-6781 (1982) on expression of bacterial CAT sequences in CV-1 monkey kidney cells, chicken embryo fibroblasts, Chinese hamster ovary cells, HeLa cells, and mouse NIH-3T3 cells using the Rous sarcoma virus long terminal repeat as a promoter.

Transcription of a DNA encoding the artemin of this invention by higher eukaryotes is often increased by inserting an enhancer sequence into the vector. Enhancers are cis-acting elements of DNA, usually about from 10 to 300 bp, that act on a promoter to increase its transcription. Enhancers are relatively orientation and position independent, having been found 5′ (Laimins et al., Proc. Natl. Acad. Sci. USA, 78:993 (1981)) and 3′ (Lusky et al., Mol Cell Bio., 3:1108 (1983)) to the transcription unit, within an intron (Banerji et al., Cell, 33:729 (1983)), as well as within the coding sequence itself. Osborne et al., Mol. Cell Bio., 4:1293 (1984). Many enhancer sequences are now known from mammalian genes (globin, elastase, albumin, fetoprotein, and insulin). Typically, however, one will use an enhancer from a eukaryotic cell virus. Examples include the SV40 enhancer on the late side of the replication origin (bp. 100-270), the cytomegalovirus early promoter enhancer, the polyoma enhancer on the late side of the replication origin, and adenovirus enhancers. See also Yaniv, Nature, 297:17-18 (1982) on enhancing elements for activation of eukaryotic promoters. The enhancer may be spliced into the vector at a position 5′ or 3′ to the artemin-encoding sequence, but is preferably located at a site 5′ from the promoter.

Expression vectors used in eukaryotic host.cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain. nucleotide segments transcribed as polyadenylated fragments in the untranslated portion of the mRNA encoding artemin.

Construction of suitable vectors containing one or more of the above-listed components employs standard ligation techniques. Isolated plasmids or DNA fragments are cleaved, tailored, and religated in the form desired to generate the plasmids required.

For analysis to confirm correct sequences in plasmids constructed, the ligation mixtures are used to transform E. coli K12 strain 294 (ATCC 31,446) and successful transformants selected by ampicillin or tetracycline resistance where appropriate. Plasmids from the transformants are prepared, analyzed by restriction endonuclease digestion, andlor sequenced by the method of Messing et al., Nucleic Acids Res., 9:309 (1981) or by the method of Maxam et al., Methods in Enzymology, 65:499 (1 980).

Particularly useful in the preparation of artemin and artemin variants are expression vectors that provide for the transient expression in mammalian cells of DNA encoding artemin. In general, transient expression involves the use of an expression vector that is able to replicate efficiently in a host cell, such that the host cell accumulates many copies of the expression vector and, in turn, synthesizes high levels of a desired polypeptide encoded by the expression vector. Sambrook et al., supta, pp. 16.17-16.22. Transient expression systems, comprising a suitable expression vector and a host cell, allow for the convenient positive identification of polypeptides encoded by cloned DNAs, as well as for the rapid screening of such polypeptides for desired biological or physiological properties. Thus, transient expression systems are particularly useful in the invention for purposes of identifying analogs and variants of artemin that are biologically active artemin.

Other methods, vectors, and host cells suitable for adaptation to the synthesis of artemin in recombinant vertebrate cell culture are described in Gething et al., Nature, 293:620-625 (1981); Mantei et al., Nature, 281:40-46 (1979); EP 117,060; and EP 117,058. A particularly useful plasmid for mammalian cell culture expression of artemin is pRK5 (EP 307,247) or pSVI6B. WO 91/08291 published 13 Jun. 1991.

Suitable host cells for cloning or.expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Kliebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DO 266,710 published 12 Apr. 1989), Pseudomonas such as P. aeruginosa, and Streptomyces. One preferred E. coli cloning host is E. coli 294 (ATCC 31,446), although other strains such as E. coi B, E. coil X1776 (ATCC 31,537), and E. coli W3110 (ATCC 27,325) are suitable. These examples are illustrative rather than limiting. Strain W3110 is a particularly preferred host or parent host because it is a common host strain for recombinant DNA product fermentations. Preferably, the host cell should secrete minimal amounts of proteolytic enzymes. For example, strain W3110 may be modified to effect a genetic mutation in the genes encoding proteins, with examples of such hosts including E. coli W3110 strain 27C7. The complete genotype of 27C7 is tonA ptr3phoA E15 (argF-lac)169 ompT degP41kan. Strain 27C7 was deposited on 30 Oct. 1991 in the American Type Culture Collection as ATCC No. 55,244. Alternatively, the strain of E. coli having mutant periplasmic protease disclosed in U.S. Pat. No. 4,946,783 issued 7 Aug. 1990 may be employed. Alternatively still, methods of cloning, e.g., PCR or other nucleic acid polymerase reactions, are suitable.

In addition to prokaryotes, eukaryotic microbes such as filamentous fungi or yeast are suitable cloning or expression hosts for arteminencoding vectors. Saccharomyces cerelvisiae, or common baker's yeast, is the most commonly used among lower eukaryotic host microorganisms. However, a number of other genera, species, and strains are commonly available and useful herein, such as Schizosaccharomyces pombe (Beach et al., Nature, 290:140 (1981); EP 139,383 published 2 May 1985); Kluyveromyces hosts (U.S. Pat. No. 4,943,529; Fleer et al., supra) such as, e.g., K. lactis (MW98-8C, CBS683, CBS4574; Louvencourt et al., J. Bacteriol., 737 (1983)), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., supra), K. thermotolerans, and K. marxianus; yarrowia (EP 402,226); Pichia pastoris (EP 183,070; Sreekrishna et al., J. Basic Microbiol., 28:265-278 (1988)); Candida; Trichoderma reesia (EP 244,234); Neurospora crassa (Case et al., Proc. Natl. Acad. Sci. USA, 76:5259-5263 (1979)); Schwanniomyces such as Schwanniomyces occidentals (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., Biochem. Biophys. fles. Commun., 1 12:284-289 (1983); Tilburn et al., Gene, 26:205-221 (1983); Yelton et al., Proc. Natl. Acad. Sci. USA, 81:1470-1474 (1984)) and A. niger. Kelly et al., EMBO J., 4:475-479 (1985).

Suitable host cells for the expression of glycosylated artemin are derived from multicellular organisms. Such host cells are capable of complex processing and glycosylation activities. In principle, any higher eukaryotic cell culture is workable, whether from vertebrate or invertebrate culture. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. See, e.g., Luckow et al., Bio/Technology, 6:47-55 (1988); Miller et al., in Genetic Engineering, Setlow et al., eds., Vol. 8 (Plenum Publishing, 1986), pp. 277-279; and Maeda et al., Nature, 315:592-594 (1985). A variety of viral strains for transfection are publicly available, e.g., the L1 variant of Autographa californica NPV and the Bm-5 strain of Bombyx mori NPV, and such viruses may be used as the virus herein according to the present invention, particularly for transfection of Spodoptera frugiperda cells.

Plant cell cultures of cotton, corn, potato, soybean, petunia, tomato, and tobacco can be utilized as hosts. Typically, plant cells are transfected by incubation with certain strains of the bacterium Agrobacterium tumefaciens, which has been previously manipulated to contain the artemin-encoding DNA. During incubation of the plant cell culture with A. tumefaciens, the DNA encoding the artemin is transferred to the plant cell host such that it is transfected, and will, under appropriate conditions, express the artemin-encoding DNA. In addition, regulatory and signal sequences compatible with plant cells are available, such as the nopaline synthase promoter and polyadenylation signal sequences. Depicker et al., J. Mol. Appl Gen., 1:561 (1982). In addition, DNA segments isolated from the upstream region of the T-DNA 780 gene are capable of activating or increasing transcription levels of plant-expressible genes in recombinant DNAcontaining plant tissue. EP 321,196 published 21 Jun. 1989.

However, interest has been greatest in vertebrate cells, and propagation of vertebrate cells in culture (tissue, culture) has become a routine procedure. See, e.g., Tissue Culture, Academic Press, Kruse and Patterson, editors (1973). Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); baby hamster kidney cells (BHK; ATCC CCL 10); Chinese hamster ovary cells/DHFR (CHO, Urlaub et al., Proc. Natl. Acad. Sci. USA, 77:4216 (1980)); mouse sertoli cells (TM4, Mather, Biol Reprod., 23:243-251 (1980)); monkey kidney cells (CVl ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (Wl38, ATCC CCL 75); human liver cells (Hep G2, HB 8065); mouse mammary tumor (MMT 060562, ATCC CCL51); TRI cells (Mather et al., Annals, N.Y. Acad. Sci., 383:44-68 (1982)); MRC 5 cells; FS4 cells; and a human hepatoma line (Hep G2).

Host cells are transfected and preferably transformed with the above-described expression or cloning vectors for artemin production and cultured in conventional nutrient media modified as appropriate for inducing promoters, selecting transformants, or amplifying the genes encoding the desired sequences.

Transfection refers to the taking up of an expression vector by a host cell whether or not any coding sequences are in fact expressed. Numerous methods of transfection are known to the ordinarily skilled artisan; for example, CaPO₄ and electroporation. Successful transfection is generally recognized when any indication of the operation of this vector occurs within the host cell.

Transformation means introducing DNA into an organism so that the DNA is replicable, either as an extrachromosomal element or by chromosomal integrant. Depending on the host cell used, transformation is done using standard techniques appropriate to such cells. The calcium treatment employing calcium chloride, as described in section 1.82 of Sambrook et al., supra, or electroporation is generally used for prokaryotes or other cells that contain substantial cell-wall barriers. Infection with Agrobacterium tomefaciens is used for transformation of certain plant cells, as described by Shaw et al., Gene, 23:315 (1983) and WO 89/05859 published 29 Jun. 1989. In addition, plants may be transfected using ultrasound treatment as described in WO 91/00358 published 10 Jan. 1991.

For mamrnalian cells without such cell walls, the calcium phosphate precipitation method of Graham et al., Virology, 52:456457(1978) is preferred. General aspects of mammalian cell host system transformations have been described in U.S. Pat. No. 4,399,216 issued 16 Aug. 1983. Transformations into yeast are typically carried out according to the method of Van Solingen et al., J. Bact., 130:946 (1977) and Hsiao et al., Proc. Natl Acad. Sci USA, 76:3829 (1979). However, other methods for introducing DNA into cells, such as by nuclear microinjection, electroporation, bacterial protoplast fusion with intact cells, or polycations, e.g., polybrene, polyomithine, etc., may also be used. For various techniques for.transforming mammalian cells, see Keown et al., Methods in Enzymology, 185:527-537 (1990) and Mansour et al., Nature, 336:348-352 (1988).

Prokaryotic cells used to produce the artemin polypeptide of this invention are cultured in suitable media as described generally in Sambrook et al., supra.

The mammalian host cells used to produce the artemin of this invention may be cultured in a variety of media. Commercially available media such as Ham's F10 (Sigma), Minimal Essential Medium ((MEM), Sigma), RPMI-1640 (Sigma), and Dulbecco's Modified Eagle's Medium ((DMEM), Sigma) are suitable for culturing the host cells. In addition, any of the media described in Ham et al., Meth. Enz., 58:44 (1979), Barnes at al., Anal. Biochem.,102:255 (1980), U.S. Pat. Nos. 4,767,704; 4,657,866; 4,927,762; 4,560,655; or 5,122,469; WO 90/03430; WO 87/00195; or U.S. Patent Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

In general, principles, protocols, and practical techniques for maximizing the productivity of mammalian cell cultures can be found in Mammalian Cell Biotechnology: a Practical Approach, M. Butler, ed. (IRL Press, 1991).

The host cells referred to in this disclosure encompass cells in culture as well as cells that are within a host animal.

Gene amplification andlor expression may be measured in a sample directly, for example, by conventional Southern blotting, Northern blotting to quantitate the transcription of mRNA (Thomas, Proc. Natl. Acad Sci. USA, 77:5201-5205 (1980)), dot blotting (DNA analysis), or in situ hybridization, using an appropriately labeled probe, based on the sequences provided herein. Various labels may be employed, most commonly radioisotopes, particularly ³²P. However, other techniques may also be employed, such as using biotin-modified nucleotides for introduction into a polynucleotide. The biotin then serves as the site for binding to avidin or antibodies, which may be labeled with a wide variety of labels, such as radionuclides, fluorescers, enzymes, or the like. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, can be measured by immunological methods, such as immunohistochemical staining of tissue sections and assay of cell culture or body fluids, to quantitate directly the expression of gene product. With immunohistochemical staining techniques, a cell sample is prepared, typically by dehydration and fixation, followed by reaction with labeled antibodies specific for the gene product coupled, where the labels are usually visually detectable, such as enzymatic labels,. fluorescent labels, luminescent labels, and the like. A particularly sensitive staining technique suitable for use in the present invention is described by Hsu et al., Am. J. Clin. Path., 75:734738 (1980).

Antibodies useful for immunohistochemical staining and/or assay of sample fluids may be either monoclonal or polyclonal, and may be prepared as described herein.

Artemin preferably is recovered from the culture medium as a secreted polypeptide, although it also may be recovered from host cell lysates. If the artemin is membrane-bound, it can be released from the membrane using a suitable detergent solution (e.g. Triton-X 100).

When artemin is produced in a recombinant cell other than one of human origin, the artemin is completely free of proteins or polypeptides of human origin. However, it is necessary to purify artemin from recombinant cell proteins or polypeptides to obtain preparations that are substantially homogeneous as to artemin. As a first step, the culture medium or lysate can be centrifuged to remove particulate cell debris. Artemin can then be purified from contaminant soluble proteins and polypeptides with the following procedures, which are exemplary of suitable purification procedures: by fractionation on an ion-exchange column; ethanol precipitation; reverse phase HPLC; chromatography on silica; chromatofocusing; immunoaffinity; epitope-tag binding resin; SOS-PAGE; ammonium sulfate precipitation; gel filtration using, for example, Sephadex G-75; and protein A Sepharose columns to remove contaminants such as IgG.

3.Modifications of Artemin

Covalent modifications of artemin and artemin variants are included within the scope of this invention. One type of covalent modification includes reacting targeted amino acid residues of an artemin polypeptide with an organic derivatizing agent that is capable of reacting with selected side chains or the N- or C-terminal residues of the artemin. Derivatization with bifunctional agents is useful, for instance, for crosslinking artemin to a water-insoluble support matrix or surface for use in the method for purifying anti-artemin antibodies, and vice versa. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W. H. Freeman & Co., San Francisco, pp. 79-86 (1983)), acetylation of the N-terminal amine, and amidation of any C-terminal carboxyl group.

Another type of covalent modification of the artemin polypeptide included within the scope of this invention comprises altering the native glycosylation pattern of the polypeptide. “Altering the native glycosylation pattern” is intended for purposes herein to mean deleting one or more carbohydrate moieties found in native sequence artemin (either by removing the underlying glycosylation site or by deleting the glycosylation by chemical andlor enzymatic means), andlor adding one or more glycosylation sites that are not present in the native sequence artemin. In addition, the phrase includes qualitative changes in the glycosylation of the native proteins, involving a change in the nature and proportions of the various carbohydrate moieties present.

Addition of glycosylation sites to the artemin polypeptide may be accomplished by altering the amino acid sequence. The alteration may be made, for example, by the addition of, or substitution by, one or more serine or threonine residues to the native sequence artemin (for O-linked glycosylation sites). The artemin amino acid sequence may optionally be altered through changes at the DNA level, particularly by mutating the DNA encoding the artemin polypeptide at pre-selected bases such that codons are generated that will translate into the desired amino acids.

Another means of increasing the number of carbohydrate moieties on the artemin polypeptide is by chemical or enzymatic coupling of glycosides to the polypeptide. Such methods are described in the art, e.g., in WO 87/05330 published 11 Sep. 1987, and in Aplin and Wriston, CRC Crit. Rev. Biochem., pp. 259-306 (1981).

Removal of carbohydrate moieties present on the artemin polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of artemin comprises linking the artemin polypeptide to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337.

The artemin of the present invention may also be modified in a way to form a chimeric molecule comprising artemin fused to another, heterologaus polypeptide or amino acid sequence.

In one embodiment, such a chimeric molecule comprises a fusion of the artemin with a tag polypeptide that provides an epitope to which an anti-tag antibody can selectively bind. The epitope tag is generally placed at the amino- or carboxyl-terminus of the artemin. The presence of such epitope-tagged forms of the artemin can be detected using an antibody against the tag polypeptide. Also, provision of the epitope tag enables the artemin to be readily purified by affinity purification using an anti-tag antibody or another type of affinity matrix that binds to the epitope tag. Various tag polypeptides and their respective antibodies are well known in the art. Examples include poly-histidine (poly-his) or poly-histidine-glycine (poly-his-gly) tags; the flue HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:12041210 (1988)]; the KT3 epitope peptide [Martin et al., Science, 255:192-194 (1992)]; an a-tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

In an alternative embodiment, the chimeric molecule may comprise a fusion of artemin with an immunoglobulin or a particular region of an immunoglobulin. For a bivalent form of the chimeric molecule (also referred to as an “immunoadhesin”), such a fusion could be to the Fc region of an IgG molecule.

The simplest and most straightforward immunoadhesin design combines the binding region(s) of the “adhesin” protein with the hinge and Fc regions of an immunoglobulin heavy chain. Ordinarily, when preparing artemin-immunoglobulin chimeras for use in the present invention, nucleic acid encoding artemin will be fused C-terminally to nucleic acid encoding the N-terminus of an immunoglobulin constant domain sequence however N-terminal fusions are also possible.

Typically, in such fusions the encoded chimeric polypeptide will retain at least functionally active hinge and CH2 and CH3 domains of the constant region of an immunoglobulin heavy chain. Fusions are also made to the C-terminus of the Fc portion of a constant domain, or immediately N-terminal to the CH1 of the heavy chain or the corresponding region of the light chain.

The precise site at which the fusion is made is not critical; particular sites are well known and may be selected in order to optimize the biological activity of the artemin-immunoglobulin chimeras.

In some embodiments, the artemin-immunoglobulin chimeras are assembled as monomers, or hetero- or homo-multimer, and particularly as dimers or tetramers, essentially as illustrated in WO 91/08298.

In a preferred embodiment, the artemin sequence is fused to the N-terminus of the C-terminal portion of an antibody (in particular the Fc domain), containing the effector functions of an immunoglobulin, e.g. immunoglobulin G₁ (IgG1). It is possible to fuse the entire heavy chain constant region to the artemin sequence. However, more preferably, a sequence beginning in the hinge region just upstream of the papain cleavage site (which defines IgG Fc chemically; residue 216, taking the first residue of heavy chain constant region to be 114, or analogous sites of other immunoglobulins) is used in the fusion. In a particularly preferred ermbodiment, the artemin amino acid sequence is fused to the hinge region and CH2 and CH3, or to the CH1, hinge, CH2 and CH3 domains of an IgG1, IgG2, or IgG3 heavy chain. The precise site at which the fusion is made is not critical, and the optimal site can be determined by routine experimentation.

In some embodiments, the artemin-immunoglobulin chimeras are assembled as multimer, and particularly as homo-dimers or tetramers. Generally, these assembled immunoglobulins will have known unit structures. A basic four chain structural unit is the form in which IgG, IgD, and IgE exist. A four-unit is repeated in the higher molecular weight immunoglobulins; IgM generally exists as a pentamer of basic four-units held together by disulfide bonds. IgA globulin, and occasionally IgG globulin, may also exist in multimeric form in serum. In the case of a multimer, each four-unit may be the same or different.

Alternatively, the artemin sequence can be inserted between immunoglobulin heavy chain and light chain sequences such that an immunoglobulin comprising a chimeric heavy chain is obtained. In this embodiment, the artemin sequence is fused to the 3′ end of an immunoglobulin heavy chain in each arm of an immunoglobulin, either between the hinge and the CH2 domain, or between the CH2 and CH3 domains. Similar constructs have been reported by Hoogenboom et al., Mol. Immunol., 28:1027-1037(1991).

Although the presence of an immunoglobulin light chain is not required in the immunoadhesins of the present invention, an immunoglobulin light chain might be present either covalently associated to an artemin-immunoglobulin heavy chain fusion polypeptide, or directly fused to artemin. In the former case, DNA encoding an immunoglobulin light chain is typically coexpressed with the DNA encoding the artemin-immunoglobulin heavy chain fusion protein. Upon secretion, the hybrid heavy chain and the light chain will be covalently associated to provide an immunoglobulin-like structure comprising two disulfide-linked immunoglobulin heavy chain-light chain pairs. Methods suitable for the preparation of such structures are, for example, disclosed in U.S. Pat. No. 4,816,567 issued 28 Mar. 1989.

In a preferred embodiment, the immunoglobulin sequences used in the construction of the immunoadhesins of the present invention are from an IgG immunoglobulin heavy chain constant domain. For human immunoadhesins, the use of human IgG1 and IgG3 immunoglobulin sequences is preferred. A major advantage of using IgG1 is that IgG1 immunoadhesins can be purified efficiently on immobilized protein A. In contrast, purification of IgG3 requires protein G, a significantly less versatile medium. However, other structural and functional properties of immunoglobulins should be considered when choosing the Ig fusion partner for a particular immunoadhesin construction. For example, the IgG3 hinge is longer and more flexible, so it can accommodate larger adhesin domains that may not fold or function properly when fused to IgG1. Another consideration may be valency; IgG immunoadhesins are bivalent homodimers, whereas Ig subtypes like IgA and IgM may give rise to dimeric or pentameric structures, respectively, of the basic Ig homodimer unit. For artemin immunoadhesins designed for ij vivo application, the pharmacokinetic properties and the effector functions specified by the Fc region are important as well. Although IgG1, IgG2 and IgG4 all have in vivo half-lives of 21 days, their relative potencies at activating the complement system are different. IgG4 does not activate complement, and IgG2 is significantly weaker at complement activation. than IgG1. Moreover, unlike IgG1, IgG2 does not bind to Fc receptors on mononuclear cells or neutrophils. While IgG3 is optimal for complement activation, its in vivo half-life is approximately one third of the other IgG isotypes. Another important consideration for immunoadhesins designed to be used as human therapeutics is the number of allotypic variants of the particular isotype. In general, IgG isotypes with fewer serologically-defined allotypes are preferred. For example, IgG1 has only four serologically-defined allotypic sites, two of which (G1m and 2) are located in the Fc region; and one of these sites G1m1, is non-immunogenic. In contrast, there are 12 serologically-defined allotypes in IgG3, all of which are in the Fc region; only three of these sites (G3m5, 11 and 21) have one allotype which is nonimmunogenic. Thus, the potential immunogenicity of a 3 immunoadhesin is greater than that of a 1 immunoadhesin.

With respect to the parental immunoglobulin, a useful joining point is just upstream of the cysteines of the hinge that form the disulfide bonds between the two heavy chains. In a frequently used design, the codon for the C-terminal residue of the artemin part of the molecule is placed directly upstream of the codons for the sequence DKTHTCPPCP of the IgG1 hinge region.

The general methods suitable for the construction and expression of immunoadhesins are the same as those disclosed hereinabove with regard to artemin. Artemin immunoadhesins are most conveniently constructed by fusing the cONA sequence encoding the artemin portion in-frame to an Ig cDNA sequence. However, fusion to genomic Ig fragments can also be used (see, e.g., Gascoigne et al., Proc. Natl. Acad. Sci. USA, 84:2936-2940 (1987); Aruffo et al., Cell, 61:1303-1313 (1990); Stamenkovic et al., Cell, 66:1133-1144 (1991)). The latter type of fusion requires the presence of Ig regulatory sequences for expression. cDNAs encoding IgG heavy-chain constant regions can be isolated based on published sequence from cDNA libraries derived from spleen or peripheral blood lymphocytes, by hybridization or by polymerase chain reaction (PCR) techniques. The cDNAs encoding the arterhin and Ig parts of the immunoadhesin are inserted in tandem into a plasmid vector that directs efficient expression in the chosen host cells. For expression in mammalian cells, pRK5-based vectors (Schall et al., Cell, 61:361-370 (1990)) and CDM8-based vectors (Seed, Nature, 329:840 (1989)) can be used. The exact junction can be created by removing the extra sequences between the designed junction codons using oligonucleotide-directed deletional mutagenesis (Zoller et al., Nucleic Acids Res., 10:6487 (1982); Capon et al., Nature, 337:525-531 (1989)). Synthetic oligonucleotides can be used, in which each half is complementary to the sequence on either side of the desired junction; ideally, these are 36 to 48-mers. Alternatively, PCR techniques can be used to join the two parts of the molecule in-frame with an appropriate vector.

The choice of host cell line for the expression of artemin immunoadhesins depends mainly on the expression vector. Another consideration is the amount of protein that is required. Milligram quantities often can be produced by transient transfections. For example, the adenovirus EIA-transformed 293 human embryonic kidney cell line can be transfected transiently with pRK5-based vectors by a modification of the calcium phosphate method to allow efficient immunoadhesin expression. CDM8-based vectors can be used to transfect COS cells by the DEAE-dextran method (Aruffo et al., Cell, 61:1303-1313 (1990); Zettmeissl et al., DNA Cell Biol. US, 9:347-353 (1990)). If larger amounts of protein are desired, the immunoadhesin can be expressed after stable transfection of a host cell line. For example, a pRK5-based vector can be introduced into Chinese hamster ovary (CHO) cells in the presence of an additional plasmid encoding dihydrofolate reductase (DHFR) and conferring resistance to G418. Clones resistant to G418 can be selected in culture; these clones are grown in the presence of increasing levels of DHFR inhibitor methotrexate; clones are selected, in which the number of gene copies encoding the DHFR and immunoadhesin sequences is co-amplified. If the immunoadhesin contains a hydrophobic leader sequence at its N-terminus, it is likely to be processed and secreted by the transfected cells. The expression of immunoadhesins with more complex structures may require uniquely suited host cells; for example, components such as light chain or J chain may be provided by certain myeloma or hybridoma cell hosts (Gascoigne et al., 1987, supra, Martin et al., J. Virol., 67:3561-3568 (1993)).

Immunoadhesins can be conveniently purified by affinity chromatography. The suitability of protein A as an affinity ligand depends on the species and isotype of the immunoglobulin Fc domain that is used in the chimera. Protein A can be used to purify immunoadhesins that are based on human 1, 2, or 4 heavy chains (Lindmark et al., J. Immunol. Meth., 62:1-13 (1983)). Protein G is recommended for all mouse isotypes and for human 3 (Guss et al., EMBO J., 5:1567-1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. The conditions for binding an immunoadhesin to the protein A or G affinity column are dictated entirely by the characteristics of the Fc domain; that is, its species and isotype. Generally, when the proper ligand is chosen, efficient binding occurs directly from unconditioned culture fluid. One distinguishing feature of immunoadhesins is that, for human 1 molecules, the binding capacity for protein A is somewhat diminished relative to an antibody of the same Fc type. Bound immunoadhesin can be efficiently eluted either at acidic pH (at or above 3.0), or in a neutral pH buffer containing a mildly chaotropic salt. This affinity chromatography step can result in an immunoadhesin preparation that is >95% pure.

Other methods known in the art can be used in place of, or in addition to, affinity chromatography on protein A or G to purify immunoadhesins. Immunoadhesins behave similarly to antibodies in thiophilic gel chromatography (Hutchens et al., Anal. Biochem., 159:217-226 (1986)) and immobilized metal chelate chromatography (Al-Mashikhi et al., J. Dairy Sci., 71:1756-1763 (1988)). In contrast to antibodies, however, their behavior on ion exchange columns is dictated not only by their isoelectric points, but also by a charge dipole that may exist in the molecules due to their chimeric nature.

If desired, the immunoadhesins can be made bispecific. Thus, the immunoadhesins of the present invention may combine an artemin domain and a domain, such as a domain from another cytokine or neurotrophic factor. For bispecific molecules, trimeric molecules, composed of a chimeric antibody heavy chain in one arm and a chimeric antibody heavy chain-light chain pair in the other arm of their antibody-like structure are advantageous, due to ease of purification. In contrast to antibody-producing quadromas traditionally used for the production of bispecific immunoadhesins, which produce a mixture of ten tetramers, cells transfected with nucleic acid encoding the three chains of a trimeric immunoadhesin structure produce a mixture of only three molecules, and purification of the desired product from this mixture is correspondingly easier.

4. Preparation and Identification of Artemin Agonists
a. Small molecule screening

Small molecules may have the ability to act as artemin agonists and thus to be useful in the prevention and treatment of nerve cell injury. Such small molecules may include naturally occurring small molecules, synthetic organic or inorganic compounds and peptides. However, small molecules in the present invention are not limited to these forms. Extensive libraries of small molecules are commercially available and a wide variety of assays are well known in the art to screen these molecules for the desired activity.

Candidate artemin agonist small molecules are preferably first identified in an assay that allows for the rapid identification of potential agonists. An example of such an assay is a protein-protein binding assay wherein the ability of the candidate molecule to bind to the GFR receptor is measured. In another example, the ability of candidate molecules to interfere with artemin binding to GFR is measured.

In a preferred embodiment, small molecule artemin agonists are identified by their ability to mimic one or more of the biological activities of artemin. For example, small molecules are screened for their ability to support the survival of dopaminergic midbrain neurons in culture, as described in Baloh et al., Neuron 211291-1302 (1998). They may also be screened for their ability to prevent axotomy induced substance P loss in the dorsal horn of the spinal cord following axotomy, as described in Example 6, without producing pain or hyperalgesia, as measured in the methods given in Examples 1 through 4.

Compounds identified as artemin agonists may be used in the methods of the present invention, for example to protect neurons in a mammal from injury-induced pathological changes.
b. Preparation and identification of agonist antibodies

Agonist human and non-human polyclonal and monoclonal antibodies (including humanized forms of non-human monoclonal antibodies) which mimic the biological properties of artemin are also contemplated in the present invention. These include amino acid sequence variants, glycosylation variants and fragments of antibodies. General techniques for the production of such antibodies and the selection of agonist antibodies are known in the art and are briefly described below.

(i) Polyclonal Antibodies

Methods of preparing polyclonal antibodies are known in the art. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. It may be useful to conjugate the immunizing agent to a protein known to be immunogenic in the mammal being immunized, such as serum albumin, or soybean trypsin inhibitor. Examples of adjuvants that may be employed include Freund's complete adjuvant and MPL-TDM.

(ii) Monoclonal Antibodies

Monoclonal antibodies may be made using the hybridoma method first described by Kohler at al., Nature, 256:495 (1975), or may be made by recombinant DNA methods (U.S. Pat. No. 4,816,567).

In the hybridoma method, a mouse or other appropriate host animal, such as a hamster or macaque monkey, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp.59-103, (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), conditions under which the growth of HGPRT-deficient cells is prevented.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOP-21 and M.C.-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001(1984); Brodeur et al., Monoclonal Antibody Production Techniques and Applications, pp.51-63, Marcel Dekker, Inc., New York, (1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the Scatchard analysis of Munson et al., Anal. Biochem., 107:220 (1980).

After hybridoma cells are identified that produce antibodies of the desired specificity, affinity, and/or activity, the cells may be subcloned by limiting dilution procedures and grown by standard methods (Goding, Monoclonal Antibodies: Principles and Practice, pp.59-103 (Academic Press, 1986)). Suitable culture media for this purpose include, for example, OMEM or RPMI1640 medium. In addition, the hybridoma cells may be grownin vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences, Morrison, et al., Proc. Nat. Acad. Sci U.S.A., 81:6851 (1984), or by covalently joining to the immunoglobulin coding sequence all or part of the coding sequence for a non-immunoglobulin polypeptide. In that manner, “chimeric” or “hybrid” antibodies are prepared that have the binding specificity of an artemin agonist monoclonal antibody described herein.

Chimeric or hybrid antibodies also may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4.mercaptobutyrimidate.

Recombinant production of antibodies will be described in more detail below.

(iii) Humanized Antibodies

Generally, a humanized antibody has one or more amino acid residues introduced into it from a non-human source. These non-human amino acid residues are often referred to as “import” residues, which are typically taken from an “import” variable domain. Humanization can be essentially performed following the method of Winter and co-workers (Jones et al., Nature, 321:522525 (1986); Riechmann et al., Nature, 332:323327 (1988); Verhoeyen et al., Science, 239:1534-1536 (1988)), by substituting rodent CDRs or CDR sequencesfor the corresponding sequences of a human antibody.

Accordingly, such “humanized” antibodies are chimeric antibodies (Cabilly, supra), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

It is important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e. the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding. For further details see U.S. application Ser. No. 071934,373 filed 21 Aug. 192, which is a continuation-in-part of application Ser. No. 07/715,272 filed 14 Jun. 1991.

(iv) Human Antibodies

Human monoclonal antibodies can be made by the hybridoma method. Human myeloma and mouse-human heteromyeloma cell lines for the production of human monoclonal antibodies have been described, for example, by Kozbor, J. Immunol. 133, 3001 (1984), and Brodeur, et al., Monoclonal Antibody Production Techniques and Applications, pages 51-63 (Marcel Dekker, Inc., New York, 1987).

It is now possible to produce transgenic animals (e.g. mice) that are capable, upon immunization, of producing a repertoire of human antibodies in the absence of endogenous immunoglobulin production. For example, it has been described that the homozygous deletion of the antibody heavy chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g. Jakobovits et al., Proc. Natl Acad. Sci. USA 90, 2551-255 (1993); Jakobovits et al., Nature 362, 255-258 (1993).

Mendez et al., (Nature Genetics 15:146-156 (1997)) have further improved the technology and have generated a line of transgenic mice designated as “Xenomouse II” that, when challenged with an antigen, generates high affinity fully human antibodies. This was achieved by germ-line integration of megabase human heavy chain and light chain loci into mice with deletion into endogenous J_{H} segment as described above. The Xenomouse II harbors 1,020 kb of human heavy chain locus containing approximately 66 V_{H} genes, complete D_{H} and J_{H} regions and three different constant regions (μ, δ and χ), and also harbors 800 kb of human κ locus containing 32 Vκ genes, Jκ segments and Cκ genes. The antibodies produced in these mice closely resemble that seen in humans in all respects, including gene rearrangement, assembly, and repertoire. The human antibodies are preferentially expressed over endogenobs antibodies due to deletion in endogenous J_{H} segment that prevents gene rearrangement in the murine locus.

Alternatively, the phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats; for their review see, e.g. Johnson, Kevin S. and Chiswell, David J., Current Opinion in Structural Biology 3:564-571 (1993). Several sources of V-gene segments can be used for phage display. Clackson et al., Nature 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including selfantigens) can be isolated essentially following the techniques described by Marks et al., J. Mol. Biol. 222:581-597 (1991), or Griffith at al., EMBO J. 12:725-734 (1993). In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen.challenge. This natural process can be mimicked by employing the technique known as “chain shuffling” (Marks et al., Bio/Technol. 10:779-783 [1992]). In this method, the affinity of “primary” human antibodies obtained by phage display can be improved by sequentially replacing the heavy and light chain V region genes with repertoires of naturally occurring variants (repertoires) of V domain genes obtained from unimmunized donors. This technique allows the production of antibodies and antibody fragments with affinities in the nM range. A strategy for making very large phage antibody repertoires (also known as “the mother-of-all libraries”) has been described by Waterhouse et al., Nuci. Acids Res. 21:2265-2266 (1993), and the isolation of a high affinity human antibody directly from such large phage library is reported by Griffith at al, EMBO J. (1994), in press. Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable domains capable of restoring a functional antigen-binding site, i.e. the epitope governs (imprints) the choice of partner. When the process is repeated in order to replace the remaining rodent V domain, a human antibody is obtained (see PCT patent application WO 93/06213, published 1 Apr. 1993). Unlike traditional humanization of rodent antibodies by COR grafting, this technique provides completely human antibodies, which have no framework or CDR residues of rodent origin.

(v) Bispecific Antibodies

Bispecific antibodies are monoclonal, preferably human or humanized, antibodies that have binding specificities for at least two different antigens. In the present case, one of the binding specificities is for the artemin receptor to provide an agonist antibody, the other one is for any other antigen, and preferably for another receptor or receptor subunit.

Methods for making bispecific antibodies are known in the art. Traditionally, the recombinant production of bispecific antibodies is based on the coexpression of two immunoglobulin heavy chain-light chain pairs, where the two heavy chains have different specificities (Millstein and Cuello, Nature 305, 537-539 (1983)). Because of the random assortment of immunoglobulin heavy and light chains, these hybridomas (quadromas) produce a potential mixture of different antibody molecules, of which only one has the correct bispecific structure. The purification of the correct molecule, which is usually done by affinity chromatography steps, is rather cumbersome, and the product yields are low. Similar procedures are disclosed in PCT application publication No. WO 93/08829 (published 13 May 1993), and in Traunecker et al., EMBO 10, 3655-3659 (1991).

According to a different and more preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2 and CH3 regions. It is preferred to have the first heavy chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are cotransfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance. In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in PCT Publication No. WO 94/04690, published on March 3, 1994.

For further details of generating bispecific antibodies see, for example, Suresh et al., Methods in Enzymology 121, 210 (1986).

(vi) Heteroconjugate Antibodies

Heteroconjugate antibodies are composed of two covalently joined antibodies. Such antibodies have, for example, been proposed to target immune system cells to unwanted cells (U.S. Pat. No. 4,676,980), and for treatment of HIV infection (PCT application publication Nos. WO 91/00360 and WO 92/200373; EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

(vii) Antibody Fragments

In certain embodiments, the artemin agonist antibody (including murine, human and humanized antibodies and antibody variants) is an antibody fragment. Various techniques have been developed for the production of antibody fragments. Traditionally, these fragments were derived via proteolytic digestion of intact antibodies (see, e.g., Morimoto et al., J. Biochem. Biophys. Methods 24:107-117 (1992) and Brennan et al., Science 229:81 (1985)). However, these fragments can now be produced directly by recombinant host cells. For example, Fab′-SH fragments can be directly recovered from E. coil and chemically coupled to form F(ab′)z fragments (Carter et al., Bio/Technology 10:163-167 (1992)). In another embodiment, the F(ab′)₂ is formed using the leucine zipper GCN4 to promote assembly of the F(ab′)2 molecule. According to another approach, Fv, Fab or F(ab′)₂ fragments can be isolated directly from recombinant host cell culture. Other techniques for the production of antibody fragments will be apparent to the skilled practitioner.

(viii) Identification of Agonist Antibodies

Potential artemin agonist antibodies may be identified based on their interaction with the GFR 3 receptor. For example, Western blot techniques well known in the art may be used to screen a variety of antibodies for their ability to bind,GFR 3. Because artemin appears to act through GFR 3 (see Example 8), any interaction between an antibody and GFR 3 would indicate that the antibody may act as an artemin agonist.

Actual artemin agonist antibodies are identified based on their biological activity. In one embodiment, artemin agonist antibodies are identified by their ability to induce survival of neonatal dorsal root ganglion neurons in vitro, as described in Example 8.

Artemin agonist antibodies may also be identified based on their ability to prevent axotomy-induced substance P loss in the dorsal horn of the spinal cord following axotomy, as described in Example 6.

5. Screening Assays For Proteins That Interact With Artemin

Any method suitable for detecting protein-protein interactions may be employed for identifying proteins, including but not limited to transmembrane or intracellular proteins, that interact with artemin. Among the traditional methods which may be employed are co-immunoprecipitation, crosslinking and co-purification through gradients or chromatographic columns to identify proteins that interact with artemin. For such assays, the artemin component can be a full length protein, a soluble derivative thereof, a peptide corresponding to a domain of interest, or a fusion protein containing some region of artemin.

Methods may be employed which result in the simultaneous identification of genes that encode proteins capable of interacting with artemin. These methods include, for example, probing expression libraries, in a manner similar to the well known technique of antibody probing of gt11 libraries, using labeled artemin or a variant thereof.

One method which detects protein interactions in vivo, the two-hybrid system, is described in detail for illustration only and not by way of limitation. One version of this system has been described (Chien et a., 1991, Proc. Natl. Acad. Sci. USA, 8B9578-9582) and is commercially available from Clontech (Palo Alto, Calif.).

Briefly, utilizing such a system, plasmids are constructed that encode two hybrid proteins: one plasmid consists of nucleotides encoding the DNA-binding domain of a transcription activator protein fused to a nucleotide sequence encoding artemin, or a polypeptide, peptide, or fusion protein therefrom, and the other plasmid consists of nucleotides encoding the transcription activator protein's activation domain fused to a cDNA encoding an unknown protein which has been recombined into this plasmid as part of a cDNA library. The DNA-binding domain fusion plasmid and the cDNA library are transformed into a strain of the yeast Saccharomyces cerevisiae that contains a reporter gene (e.g., HBS or lacZ) whose regulatory region contains the transcription activator's binding site. Either hybrid protein alone cannot activate transcription of the reporter gene: the DNA-binding domain hybrid cannot because it does not provide activation function and the activation domain hybrid cannot because it cannot localize to the activator's binding sites. Interaction of the two hybrid proteins reconstitutes the functional activator protein and results in expression of the reporter gene, which is detected by an assay for the reporter gene product.

The two-hybrid system or related methodology may be used to screen activation domain libraries for proteins that interact with the “bait” gene product. By way of example, and not by way of limitation, artemin can be used as the bait gene product. Total genomic or cDNA sequences are fused to the DNA encoding an activation domain. This library and a plasmid encoding a hybrid of a bait artemin gene product fused to the DNA-binding domain are cotransformed into a yeast reporter strain, and the resulting transformants are screened for those that express the reporter gene. For example, and not by way of limitation, a bait artemin gene sequence, e.g., the genes open reading frame, can be cloned into a vector such that it is translationally fused to the DNA encoding the DNA-binding domain of the GAL4 protein. These colonies are purified and the library plasmids responsible for reporter gene expression are isolated. DNA sequencing is then used to identify the proteins encoded by the library plasmids.

A cDNA library of the cell line from which proteins that interact with the bait artemin gene product are to be detected can be made using methods routinely practiced in the art. According to the particular system described herein, for example, the cDNA fragments can be inserted into a vector such that they are translationally fused to the transcriptional activation domain of GAL4. This library can be co-transformed along with the bait artemin gene-GAL4 fusion plasmid into a yeast strain which contains a lacZ gene driven by a promoter which contains a GAL4 activation sequence. A cDNA encoded protein, fused to GAL4 transcriptional activation domain, that interacts with the bait artemin gene product will reconstitute an active GAL4 protein and thereby drive expression. Colonies which drive expression can be detected by methods routine in the art. The cDNA can then be purified from these strains, and used to produce and isolate the bait artemin gene-interacting protein using techniques routinely practiced in the art.

a. Assays For Compounds That Modulate Artemin Expression Or Activity

The following assays are designed to identify compounds that interact with (e.g., bind to) artemin, compounds that interfere with the interaction of artemin with its binding partners, cognate or substrate, and to compounds that modulate the activity of artemin gene expression (i.e., modulate the level of artemin gene expression) or modulate the levels of artemin in the body. Assays may additionally be-utilized which identify compounds that bind to artemin gene regulatory sequences (e.g., promoter sequences) and, consequently, may modulate artemin gene expression. See, e.g., Platt, K. A., 1994, J. Biol. Chem. 269:28558-28562, which is incorporated herein by reference in its entirety.

The compounds which may be screened in accordance with the invention include, but are not limited to peptides, antibodies and fragments thereof, and other organic compounds (e.g., peptidomimetics) that bind to an artemin or an artemin receptor and either mimic the activity triggered by a natural ligand (ie., agonists) or inhibit the activity triggered by the natural ligand (i.e. antagonists).

Such compounds may include, but are not limited to, peptides such as, for example, soluble peptides, including but not limited to members of random peptide libraries; (see, e.g., Lam, K. S. et al., 1991, Nature 354:82-84; Houghten, R. et al., 1991, Nature 354:84-86), and combinatorial chemistry-derived molecular library made of D- and/or L-configuration amino acids; phosphopeptides (including, but not limited to members of random or partially degenerate, directed phosphopeptide libraries; see, eg., Songyang, Z. et al., 1993, Cell 72:767-778), antibodies (including, but not limited to, polyclonal, monoclonal, humanized, anti-idiotypic, chimeric or single chain antibodies, and FAb, F(ab )₂ and FAb expression library fragments, and epitope-binding fragments thereof), and small organic or inorganic molecules.

Other compounds which can be screened in accordance with the invention include, but are not limited to small organic molecules that are able to cross the blood-brain barrier, gain entry into an appropriate cell (e.g., in the choroid plexus, pituitary, the hypothalamus, etc.) and affect the expression of an artemin gene or some other gene involved in an -artemin mediated pathway (e.g., by interacting with the regulatory region or transcription factors involved in gene expression); or such compounds that affect or substitute for the activity of the artemin or the activity of some other intracellular factor involved in an artemin signal transduction, catabolic, or metabolic pathways.

Computer modeling and searching technologies permit identification of compounds, or the improvement of already identified compounds, that can modulate artemin expression or activity. Having identified such a compound or composition, the active sites or regions are identified. Such active sites might typically be ligand binding sites. The active site can be identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods can be used to find the active site by finding where on the factor the complexed ligand is found.

Next, the three dimensional geometric structure of the active site is determined. This can be done by known methods, including X-ray crystallography, which can determine a complete molecular structure. On the other hand, solid or liquid phase NMR can be used to determine certain intra-molecular distances. Any other experimental method of structure determination can be used to obtain partial or complete geometric structures. The geometric structures may be measured with a complexed ligand, natural or artificial, which may increase the accuracy of the active site structure determined.

If an incomplete or insufficiently accurate structure is determined, the methods of computer based numerical modeling can be used to complete the structure or improve its accuracy. Any recognized modeling method may be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods.

Finally, having determined the structure of the active site (or binding site), either experimentally, by modeling, or by a combination, candidate modulating compounds can be identified by searching databases containing compounds along with information on their molecular structure. Such a search seeks compounds having structures that match the determined active site structure and that interact with the groups defining the active site. Such a search can be manual, but is preferably computer assisted. These compounds found from this search are potential modulators of artemin activity.

Alternatively, these methods can be used to identify improved modulating compounds from an already known modulating compound or ligand. The composition of the known compound can be modified and the structural effects of modification can he determined using the experimental and computer modeling methods described above applied to the new composition. The altered structure is then compared to the active site structure of the compound to determine if an improved fit or interaction results. In this manner systematic variations in composition, such as by varying side groups, can be quickly evaluated to obtain modified modulating compounds or ligands of improved specificity or activity.

Further experimental and computer modeling methods useful to identify modulating compounds based upon identification of the active sites (or binding sites) of an artemin, and related transduction and transcription factors will be apparent to those of skill in the art.

Examples of molecular modeling systems are the CHARMm and QUANTA programs (Polygen Corporation Waltham, Mass.). CHARMm performs the energy minimization and molecular dynamics functions. QUANTA performs the construction, graphic modeling and analysis of molecular structure. QUANTA allows interactive construction modification, visualization, and analysis of the behavior of molecules with each other.

A number of articles review computer modeling of drugs interactive with specific proteins, such as Rotivinen, et al., 1988, Acta Pharmaceutical Fennica 97:159-166; Ripka, New Scientist 54.57 (Jun. 16, 1988) McKinaly and Rossmann, 1989, Annu. Rev. Pharmacol. Toxiciol. 29:111-122; Perry and Davies, OSAR: Quantitative Structure-Activity Relatiorships in Drug Design pp.189-193 (Alan R. Liss, Inc. 1989); Lewis and Dean, 1989 Proc. R Soc. Lond. 236:125-140 and 141-162; and, with respect to a model receptor for nucleic acid components, Askew, et al., 1989, J. Am. Chem. Soc. 111:1082-1090. Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario). Although these are primarily designed for application to drugs specific to particular proteins, they can be adapted to design of drugs specific to regions of DNA or RNA, once that region is identified.

Although described above with reference to design and generation of compounds which could alter binding one could also screen libraries of known compounds, including natural products or synthetic chemicals, and biologically active materials, including proteins, for compounds which are inhibitors or activators.

Compounds identified via assays such as those described herein may be useful, for example, in elucidating the biological function of an artemin gene product. Such compounds can be administered to a patient at therapeutically effective doses to treat any of a variety of physiological or mental disorders. A therapeutically effective dose refers to that amount of the compound sufficient to result in any amelioration, impediment, prevention or alteration of any biological symptom.

b. Assays For Compounds That Bind To Artemln

Systems may be designed to identify compounds capable of interacting with (e.g., binding to) or mimicking artemin, or capable of interfering with the binding of artemin to a cognate receptor, binding partner or substrate. The compounds identified can be useful, for example, in modulating the activity of wild type and/or mutant artemin gene products; can be useful in elaborating the biological function of artemin; can be utilized in screens for identifying compounds that disrupt normal artemin interactions; or may themselves disrupt or activate such interactions.

The principle of the assays used to identify compounds that bind to artemrin, or artemiri cognate receptors of substrates, involves preparing a reaction mixture of artemin and the test compound under conditions and for a time sufficient to allow the two components to interact and bind, thus forming a complex which can be removed and/or detected in the reaction mixture. The artemin species used can vary depending upon the goal of the screening assay. For example, where agonists of the natural receptor are desired, the full length artemin, or a soluble truncate artemin, a peptide, or fusion protein containing one or more artemin domains fused to a protein or polypeptide that affords advantages in the assay system (e.g., labeling, isolation of the resulting complex, etc.) can be utilized. Where compounds that directly interact with artemin are sought, peptides corresponding to the artemin and fusion proteins containing artemin can be used.

The screening assays can be conducted in a variety of ways. For example, one method to conduct such an assay would involve anchoring the artemin, polypeptide, peptide, or fusion protein therefrom, or the test substance onto a solid phase and detecting arteminitest compound complexes anchored on the solid phase at the end of the reaction. In one embodiment of such a method, the artemin reactant may be anchored onto a solid surface, and the test compound, which is not anchored, may be labeled, either directly or indirectly.

In practice, microtiter plates may conveniently be utilized as the solid phase. The anchored component may be immobilized by non-covalent or covalent attachments. Non-covalent attachment may be accomplished by simply coating the solid surface with a solution of the protein and drying. Alternatively, an immobilized antibody, preferably a monoclonal antibody, specific for the protein to be immobilized may be used to anchor the protein to the solid surface. The surfaces may be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the previously nonimmobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for a artemin protein, polypeptide, peptide or fusion protein or the test compound to anchor any complexes formed in solution, and a labeled antibody specific for the other component of the possible complex to detect anchored complexes.

C. Assays For Compounds That Interfere With Artemin Interactions

Macromolecules that interact with artemin are referred to, for purposes of this discussion, as “binding partners”. These binding partners are likely to be involved in artemin mediated biological pathways. Therefore, it is desirable to identify compounds that interfere with or disrupt the interaction of such binding partners which may be useful in regulating or augmenting artemin activity in the body andior controlling disorders associated with this activity (or a deficiency thereof).

The basic principle of the assay systems used to identify compounds that interfere with the interaction between artemin and its binding partner or partners involves preparing a reaction mixture containing artemin, or some variant thereof, and the binding partner under conditions and for a time sufficient to allow the two to interact and bind, thus forming a complex. In order to test a compound for inhibitory activity, the reaction mixture is prepared in the presence and absence of the test compound. The test compound may be initially included in the reaction mixture, or may be added at a time subsequent to the addition of the artemin and its binding partner. Control reaction mixtures are incubated without the test compound or with a placebo. The formation of any complexes between the artemin and the binding partner is then detected. The formation of a complex in the control reaction, but not in the reaction mixture containing the test compound, indicates that the compound interferes with the interaction of the artemin and the interactive binding partner. Additionally, complex formation within reaction mixtures containing the test compound and normal artemin protein may also be compared to complex formation within reaction mixtures containing the test compound and a mutant artemin. This comparison may be important in those cases wherein it is desirable to identify compounds that specifically disrupt interactions of mutant, or mutated, artemin but not the normal proteins.

The assay for compounds that interfere with the interaction between artemin and binding partners can be conducted in a heterogeneous or homogeneous format. Heterogeneous assays involve anchoring either the artemin, or the binding partner, onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction by competition can be identified by conducting the reaction in the presence of the test substance; i.e., by adding the test substance to the reaction mixture prior to, or simultaneously with, artemin and interactive binding partner. Alternatively, test compounds that disrupt preformed complexes, e.g. compounds with higher binding constants that displace one of the components from the complex, can be tested by adding the test compound to the reaction mixture after complexes have been formed. The various formats are described briefly below.

In a heterogeneous assay system, either artemin or an interactive binding partner, is anchored onto a solid surface, while the non-anchored species is labeled, either directly or indirectly. In practice, microtiter plates are conveniently utilized. The anchored species may be immobilized by non-covalent or covalent attachments. Non-covalent attachment may be accomplished simply by coating the solid surface with a solution of the artemin or binding partner and drying. Alternatively, an immobilized antibody specific for the species to be anchored may be used to anchor the species to the solid surface. The surfaces may be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not prelabeled, an indirect label can be used to detect complexes anchored on the surface; e.g., using a labeled antibody specific for the initially non-immobilized species (the antibody, in turn, may be directly labeled or indirectly labeled with a labeled anti-Ig antibody). Depending upon the order of addition of reaction components, test compounds which inhibit complex formation or which disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds which inhibit complex or which disrupt preformed complexes can be identified.

In an alternate embodiment of the invention, a homogeneous assay can be used. In this approach, a preformed complex of artemin and an interactive binding partner is prepared in which either the artemin or its binding partners is labeled, but the signal generated by the label is quenched due to formation of the complex (see, e.g., U.S. Pat. No. 4,109,496 by Rubenstein which utilizes this approach for immunoassays). The addition of a test substance that competes with and displaces one of the species from the preformed complex will result in the generation of a signal above background. In this way, test substances which disrupt the interaction can be identified.

In a particular embodiment, an artemin fusion can be prepared for immobilization. For example, artemin, or a peptide fragment thereof, can be fused to a glutathione-S-transferase (GST) gene using a fusion vector, such as pGEX-5X-1, in such a manner that its binding activity is maintained in the resulting fusion protein. The interactive binding partner can be purified and used to raise a monoclonal antibody, using methods routinely practiced in the art and described above. This antibody can be labeled with the radioactive isotope ¹²⁵I, for example, by methods routinely practiced in the art. In a heterogeneous assay, the fusion protein can be anchored to glutathione-agarose beads. The interactive binding partner can then be added in the presence or absence of the test compound in a manner that allows interaction and binding to occur. At the end of the reaction period, unbound material can be washed away, and the labeled monoclonal antibody can be added to the system and allowed to bind to the complexed components. The interaction between artemin and the interactive binding partner can be detected by measuring the amount of radioactivity that remains associated with the glutathioneagarose beads. A successful inhibition of the interaction by the test compound will result in a decrease in measured radioactivity.

Alternatively, the GST fusion protein and the interactive binding partner can be mixed together in liquid in the absence of the solid glutathioneagarose beads. The test compound can be added either during or after the species are allowed to interact. This mixture can then be added to the glutathioneagarose beads and unbound material is washed away. Again the extent of inhibition of the interaction between artemin and the binding partner can be detected by adding the labeled antibody and measuring the radioactivity associated with the beads.

In another embodiment of the invention, these same techniques can be employed using peptide fragments that correspond to the binding domains of artemin and/or the interactive or binding partner (in cases where the binding partner is a protein), in place of one or both of the full length proteins. Any number of methods routinely practiced in the art can be used to identify and isolate the binding sites. These methods include, but are not limited to, mutagenesis of the gene encoding one of the proteins and screening for disruption of binding in a co-immunoprecipitation assay. Compensatory mutations in the gene encoding the second species in the complex can then be selected. Sequence analysis of the genes encoding the respective proteins will reveal the mutations that correspond to the region of the protein involved in interactive binding. Alternatively, one. protein can be anchored to a solid surface using methods described above, and allowed to interact with and bind to its labeled binding partner, which has been treated with a proteolytic enzyme, such as trypsin. After washing, a relatively short, labeled peptide comprising the binding domain may remain associated with the solid material, which can be isolated and identified by amino acid sequencing. Also, once the gene coding for the intracellular binding partner is obtained, short gene segments can be engineered to express peptide fragments of the protein, which can then be tested for binding activity and purified or synthesized.

For example, and not by way of limitation, an artemin can be anchored to a solid material as described, above, by making a GST fusion protein and allowing it to bind to glutathione agarose beads. The interactive binding partner can be labeled with a radioactive isotope, such as ³⁵S, and cleaved with a proteolytic enzyme such as trypsin. Cleavage products can then be added to the anchored fusion protein and allowed to bind. After washing away unbound peptides, labeled bound material, representing the intracellular binding partner binding domain, can be eluted purified, and analyzed for amino acid sequence by well-known methods. Peptides so identified can be produced synthetically or fused to appropriate facilitative proteins using recombinant DNA technology.

6. Pharmaceutical Compositions

Artemin in purified form may be entrapped in microcapsules prepared, for example, by coacervation techniques or by interfacial polymerization (for example, hydroxymethylcellulose or gelatin-microcapsules and poly (methylmethacylate) microcapsules, respectively), in colloidal drug delivery systems (for example, liposomes, albumin microspheres, microemulsions, nano-particles and nanocapsules) or in macroemulsions. Such techniques are disclose in Remington's Pharmaceutical Sciences 16th edition, 1980, (A. Osol, Ed).

Therapeutic formulations of artemin are prepared by mixing artemin having the desired degree of purity preferably essentially pure, with optional physiologically acceptable carriers, excipients or stabilizers (Remington's Pharmaceutical Sciences, supra), in the form of lyophilized cake or aqueous solutions. Acceptable carriers, excipient or stabilizers are nontoxic to the cell or mammal being exposed at the dosages and concentrations employed Examples include buffers such as phosphate, citrate and other organic acids; antioxidants including ascorbic acid; low molecular weight (less than about 10 residues) polypeptides; proteins, such as serum albumin, gelatin of immunoglobulins; hydrophilic polymers such as polyvinylpyrrolidone, amino acids such as glycine, glutamine asparagine, arginine or lysine; monosaccharides, disaccharides and other carbohydrates including glucose, mannose, of dextrins; chelating agents such as EDTA; sugar alcohols such as mannitol or sorbitol; salt-forming counterions such as sodium; andlor nonionic surfactants such as Tween, Pluronics or PEG.

Artemin to be used for in vivo administration must be sterile. This is readily accomplished by any method known in the art, such as filtration through sterile filtration membranes, prior to or following lyophilization and reconstitution. Artemin may be stored in lyophilized form. Therapeutic artemin compositions generally are placed into a container having a sterile access port, for example, an intravenous solution bag or vial having a stopper pierceably by a hypodermic injection needle.

Artemin optionally is combined with or administered in concert with other neurotrophic factors including but not limited to NGF, NT-3, andlor BDNF and is used with other conventional therapies for degenerative nervous disorders.

Artemin may be administered continuously by infusion into the fluid reservoirs of the CNS, although bolu injection is acceptable. In one embodiment, artemin preferably is administered into the ventricles of the brain of otherwise introduced into the CNS or spinal fluid. It can be administered by an indwelling catheter using a continuous administration means such as a pump, or it can be administered by implantation, e.g., intracerebral implantation, of sustained-release vehicle. More specifically, artemin can be injected through chronically implanted cannulas of chronically infused with the help of osmotic minipumps. Subcutaneous pumps are available that deliver protein through a small tubing to the cerebral ventricles. Highly sophisticated pumps can be refilled through the skin and their delivery rate can be set without surgical intervention.

Examples of suitable administration protocols and delivery systems involving a subcutaneous pump device of continuous intracerebroventricular infusion through a totally implanted drug delivery system are those used for the administration of dopamine, dopamine agonists, and cholinergic agonists to Alzheimer patients and animal models for Parkinson's disease described by Harbaugh, J. Neural Transm. Suppl., 24:271 (1987); and DeYebenes, et al., Mov. Disord., 2:143 (1987). Artemin agonist antibody is administered in the same fashion, or by administration into the blood stream or lymph.

Suitable examples of sustained release preparations include semipermeable polymer matrices in the form of shaped articles, e.g. films, or microcapsules. Examples of sustained release matrices include polyesters, hydrogels (e.g. poly(2-hydroxyethyl-methacrylate) as described by Langer et al., J. Biomed. Mater. Res., 15:167.277 (1981) and Langer, Chem. Tech., 12:98-105 (1982) or poly(vinylalcohol)), polylactides (U.S. Pat. No. 3,773,919, EP 58,481), copolymers of L-glutamic acid and gamma ethyl-L-glutamate (Sidman, et al., Biopolymers 22:547 (1983)), non-degradable ethylene vinyl acetate (Langer, et al., supra) or degradable lactic acid-glycolic acid copolymers such as the Lupron Depot™ (injectable microspheres composed of lactic acid-glycoloic acid copolymer and leuprolide acetate), and poly-D-(-)-3-hydroxybutyric acid (EP 133,988).

While polymers such as ethylene-vinyl acetate and lactic acid-glycolic acid enable release of molecules for over 100 days, certain hydrogels release proteins for shorter time periods. When encapsulated proteins remain in the body for a long time, they may denature or aggregate as a result of exposure to moisture at 37 C, resulting in a loss of biological activity and possible changes in immunogenicity. Rational strategies can be devised for protein stabilization depending on the mechanism involved. For example, if the aggregation mechanism is discovered to be intermolecular S-S bond formation through thio-disulfide interchange, stabilization may be achieved by modifying sulfhydryl residues, lyophilizing from acidic solutions, controlling moisture content, using appropriate additives, and developing specific polymer matrix compositions.

Sustained release artemin compositions also include liposomally entrapped artemin. Liposomes containing artemin are prepared by methods known in the art. (Epstein, et al., 1985, Proc. Natl. Acid. Sci. 82:3688; Hwang, et al., 1980, Proc. Natl. Acad. Sci. USA 77:4030; DE 3,218,121 A; EP 52322A; EP 36676A; EP 88046A; EP 143949A; EP 142641A; Japanese Pat. App. No. 83-118008; U.S. Pat. Nos. 4,485,045 and 4,544,545; and EP 102,324A). Ordinarily the liposomes are of the small (about 200-800 Angstroms) unilamelar type in which the lipid content is greater than about 30 mol. % cholesterol, the selected proportion being adjusted for the optimal artemin therapy.

When applied topically, artemin is suitably combined with other ingredients, such as carriers and/or adjuvants. There are no limitations on the nature of such other ingredients, except that they must be physiologically acceptable and efficacious for their intended administration, and cannot degrade the activity of the active ingredients of the composition. Examples of suitable vehicles include ointments, creams, gelsi or suspensions, with or without purified collagen. The compositions also may be impregnated into transdermal patches, plasters, and bandages, preferably in liquid or semi-liquid form.

For obtaining a gel formulation, artemin formulated in a liquid composition may be mixed with an effective amount of a water-soluble polysaccharide or synthetic polymer such as PEG to form a gel of the proper viscosity to be applied topically. The polysaccharide that may be used includes, for example, cellulose derivatives such as etherified cellulose derivatives, including alkyl celluloses, hydroxyalkyl celluloses, and alkylhydroxyalkyl celluloses, for example, methylcellulose, hydroxyethyl cellulose, carboxymethyl cellulose, hydroxypropyl methylcellulose, and hydroxypropyl cellulose; starch and fractionated starch; agar; alginic acid and alginates; gum arabic; pullullan; agarose; carrageenan; dextrans; dextrins; fructans; inulin; mannans; xylans; arabinans; chitosans; glycogens; glucans; and synthetic biopolymers; as well as gums such as xanthan gum; guar gum; locust bean gum; gum arabic; tragacanth gum; and karaya gum; and derivatives and mixtures thereof. The preferred gelling agent herein is one that is inert to biological systems, nontoxic, simple to prepare, and not too runny or viscous, and will not destabilize the artemin held within it.

Preferably the polysaccharide is an etherified cellulose derivative, more preferably one that is well defined, purified, and listed in USP, e.g., methylcellulose and the hydroxyalkyl cellulose derivatives, such as hydroxypropyl cellulose, hydroxyethyl cellulose, and hydroxypropyl methylcellulose. Most preferred herein is methylcellulose.

The polyethylene glycol useful for gelling is typically a mixture of low and high molecular weight PEGs to obtain the proper viscosity. For example, a mixture of a PEG of molecular weight 400-600 with one of molecular weight 1500 would be effective for this purpose when mixed in the proper ratio to obtain a paste.

The term “water soluble” as applied to the polysaccharides and PEGs is meant to include colloidal solutions and dispersions. In general, the solubility of the cellulose derivatives is determined by the degree of substitution of ether groups, and the stabilizing derivatives useful herein should have a sufficient quantity of such ether groups per anhydroglucose unit in the cellulose chain to render the derivatives water soluble. A degree of ether substitution of at least 0.35 ether groups per anhydroglucose unit is generally sufficient. Additionally, the cellulose derivatives may be in the form of alkali metal salts, for example, the Li, Na, K, or Cs salts.

If methylcellulose is employed in the gel, preferably it comprises about 2-5%, more preferably about 3%, of the gel and artemin is present in an amount of about 300-1000 mg per ml of gel.

Semipermeable, implantable membrane devices are useful as means for delivering drugs in certain circumstances. For example, cells that secrete artemin, artemin variants, artemin chimeras or artemin agonists can be encapsulated, and such devices can be implanted into a patient. For example, they may be implanted into the brains of patients suffering from Parkinson's Disease. See, U.S. Pat. No. 4,892,538 of Aebischer et al.; U.S. Pat. No. 5,011,472 of Aebischer et al.; U.S. Pat. No. 5,106,627 of Aebischer et al.; PCT Application WO 91110425; PCT Application WO 91/10470; Winn et al., Exper. Neurology, 113:322-329 (1991); Aebischer et al., Exper. Neurology, 111:269-275 (1991); and Tresco et al., ASAIO, 38:17-23 (1992). Accordingly, also included is a method for preventing or treating damage to a neuron which comprises implanting cells that secrete artemin, its agonists or antagonists as may be required for the particular condition, into the body of patients in need thereof. Finally, the present invention includes a device for preventing or treating nerve damage by implantation into a patient of an implant comprising a semipermeable membrane, and cells that secrete artemin (or its agonists or antagonists as may be required for the particular condition) encapsulated within said membrane and said membrane being permeable to artemin (or its agonists or antagonists) and impermeable to factors from the patient detrimental to the cells. The patient's own cells, transformed to produce artemin ex vivo, could be implanted directly into the patient, optionally without such encapsulation. The methodology for the membrane encapsulation of living cells is familiar to those of ordinary skill in the art, and the preparation of the encapsulated cells and their implantation into patients may be accomplished without undue experimentation.

The pharmaceutical composition comprising artemin or artemin agonist is preferably located in a suitable container. The container is preferably accompanied by instructions detailing the appropriate use and dosage of the pharmaceutical composition. One skilled in the art will recognize that these instructions will vary depending upon the method of treatment.

7. Methods Of Treatment

Artemin is believed to find use as an agent for enhancing the survival of nerve cells. It therefore is useful in the prevention, amelioration or treatment of degenerative disorders of the nervous system (“neurodegenerative diseases”), including but not limited to such disorders as Alzheimer's disease, Parkinson's disease, Huntington's chorea, ALS, peripheral neuropathies, and other conditions characterized by necrosis or loss of neurons, whether central, peripheral, or motorneurons. In addition, it may be useful for treating damaged nerve cells, e.g., nerves damaged by traumatic conditions such as bums and wounds, diabetes, kidney dysfunction, and the toxic effects of chemotherapeutics used to treat cancer and AIDS. In the appropriate cases it may also be useful in preventing or ameliorating nerve damage or the deleterious response to nerve damage in uninjured cells.

The present invention is based on the discovery that the benefits of artemin treatment in mammals are not accompanied by the problematic side effects seen with NGF treatment. The experiments described herein demonstrate that artemin has neuroprotective properties such as the ability to protect neurons from pathological changes associated with injury and the ability to protect neurons from viralinduced death. However, the protective effects of artemin are not accompanied by pain or hyperalgesia.

In one embodiment of the present invention, a patient with nerve damage is administered a therapeutically effective amount of artemin. A disease or medical disorder is considered to be nerve damage if the survival or function of nerve cells and/or their axonal processes is compromised. Such nerve damage occurs as the result of conditions including (a) physical injury, which causes the degeneration of the axonal processes and/or nerve cell bodies near the site of the injury; (b) ischernia, as a stroke; (c) exposure to neurotoxins, such as cancer and AIDS chemotherapeutic agents like cisplatin and dideoxycytidine (ddC), respectively; (d) chronic metabolic diseases, such as diabetes or renal dysfunction; and (e) neurodegenerative diseases such as Parkinson's disease, Alzheimer's disease, and Amyotrophic Lateral Sclerosis (ALS), which cause the degeneration of specific neuronal populations. Conditions involving nerve damage include Parkinson's disease, Alzheimer's disease, Amyotrophic Lateral Sclerosis, stroke, diabetic polyneuropathy, toxic neuropathy, and physical damage to the nervous system such as that caused by physical injury of the brain and spinal cord or crush or cut injuries to the arm and hand or other parts of the body, including temporary or permanent cessation of blood flow to parts of the nervous system, as in stroke.

The present invention includes, therefore, a method for preventing, ameliorating or treating nerve damage by implanting cells, into the body of a patient in need thereof, said cells either selected for their natural ability to generate artemin or engineered to secrete artemin. Preferably, the secreted artemin being soluble, mature human artemin when the patient is human. The implants are preferably nonimmunogenic andlor prevent immunogen implanted cells from being recognized by the immune system. For CNS delivery, a preferred location for the implant the cerebral spinal fluid of the spinal cord.

In one embodiment, the compounds identified by the screening assays in section 5, above, may be used modulate the level of artemin activity or expression. Specifically, compounds identified which are able to stimulate the binding or artemin to its receptor may be useful for treatments wherein increased level of artemin activity desired. Similarly, compounds identified which are able to increase artemin gene expression may be useful for similar treatments.

In one embodiment, a patient suffering from peripheral nerve damage, especially peripheral sensor neuropathy is treated with artemin administration. In particular it is contemplated that patients suffering from diabetic neuropathy will be treated with artemin administration.

In another embodiment, artemin is administered to a patient to protect peripheral neurons from injury induced pathological changes. However, it is also contemplated that artemin will be administered to a patient protect central and motor neurons from injury induced pathological changes.

In yet another embodiment, artemin is administered to a patient in combination with one or more chemotherapeutic agents, such as in the treatment of cancer. It is contemplated that artemin may be administered prior to, during or after treatment with the chemotherapeutic agent such that nerve damage is prevented or treated. Preferred chemotherapeutic agents include but are not limited to vincristine, cisplatin, methotrexate, 3′-azido-3deoxythymidine, taxanes (e.g. paclitaxel (TAXOL®, Bristol-Myers Squibb Oncology, Princeton, N.J.) and doxetax (TAXOTERE®, Rhône-Poulenc Rorer, Antony, France)) and/or anthracycline antibiotics. The manufacturer instructions may be followed in determining the preparation and dosing schedules for such chemotherapeutic agent or they may be determined empirically by the skilled practitioner. Preparation and dosing schedules for suitable chemotherapy are also described in Chemotherapy Service Ed., M.C. Perry, Williams & Wilkins, Baltimore, Md. (1992).

An effective amount of artemin to be employed therapeutically will depend, for example, upon the therapeutic objectives, the route of administration, and the condition of the patient. Accordingly, it will be necessary for the therapist to titer the dosage and modify the route of administration as required to obtain the optimal therapeutic effect. Typically, the clinician will administer the artemin until a dosage is reached that achieves the desired effect.

A typical daily dosage for systemic treatment might range from about 0.01 g/kg up to 50 mg/kg or more depending on the factors mentioned above. As an alternative general proposition, the artemin is formulated at delivered to the target site or tissue at a dosage capable of establishing in the tissue an artemin level that is efficacious but not unduly toxic. This intra-tissue concentration should be maintained if possible by continuous infusion, sustained release, topical application, arteminexpressing cell implant, or injection at empirically determined frequencies. The progress of this therapy is easily monitored by conventional assays.

Injury-induced pathological neuronal changes are preferably prevented or treated in a mammal by administration of from about 0.01 g/kg to about 1 mg/kg artemin or artemin agonist. In one embodiment, injury-induced neuronal changes are prevented or treated in a mammal by administration of from about 0.1 g/kg to about 1 mg/kg artemin or artemin agonist. More preferably from about 1 g/kg to about 1 mg/kg and even more preferably from about 10 g/kg to about 1 mg/kg artemin or artemin agonist are administered. In another embodiment, injury-induced neuronal changes are prevented or treated in a mammal by administration of from about 0.1 mg/kg to about 1 mg/kg. More preferably from about 0.25 to about 1 mg/kg and even more preferably from about 0.75 to about 1 mg/kg artemin or artemin agonist are administered. The route of artemin administration is in accord with known methods, e.g. injection or infusion by intravenous, intradermal, intraperitoneal, intracerebral, intramuscular, intraocular, intraarterial or intralesional routes, topical administration, or by sustained release systems. In a preferred embodiment artemin is administered by intravenous, intradermal, intrathecal or subcutaneous injection. However in another embodiment it is administered topically.

The dosing regimen must be determined based on the individual circumstances. However, in a preferred embodiment, artemin or an artemin agonist is administered every day, more preferably every other day and even more preferably at least two times a week. The treatment is preferably continued for six months, more preferably for one month and even more preferably for at least two weeks. One skilled in the art will appreciate that the exact dosing regimen must be determined by the therapist based on the individual circumstances.

In one example, artemin or an artemin agonist is administered to a patient suffering from diabetic neuropathy. A dose of between about 0.5 g/kg and 1 mg/kg is administered by intravenous injection at least three times a week for at least two weeks. The patient does not suffer pain or hyperalgesia associated with the injections.

8. Articles Of Manufacture

In another aspect the invention contemplates an article of manufacture containing materials useful for the treatment or prevention of neuronal damage. The article of manufacture comprises a container and a label or package insert on or associated with the container. Suitable containers include, for example, bottles, vials, syringes etc. The containers may be formed from a variety of materials such as glass and plastic. The container holds a composition comprising artemin and the label or package insert indicates that the composition is to be administered in a dose of between about 0.01 g/kg and 1 mg/kg. Optionally, the label may also indicate that the composition is useful for the treatment and/or prevention of neuronal damage.

### EXAMPLE 1

### Systemic NGF Causes Thermal and Mechanical Hyperalgesia While Artemin Does Not

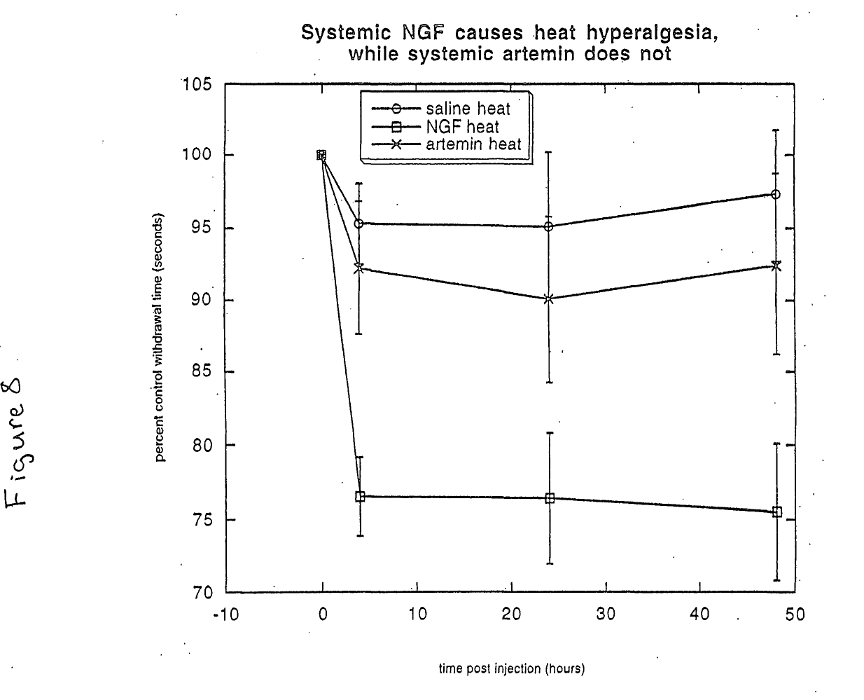

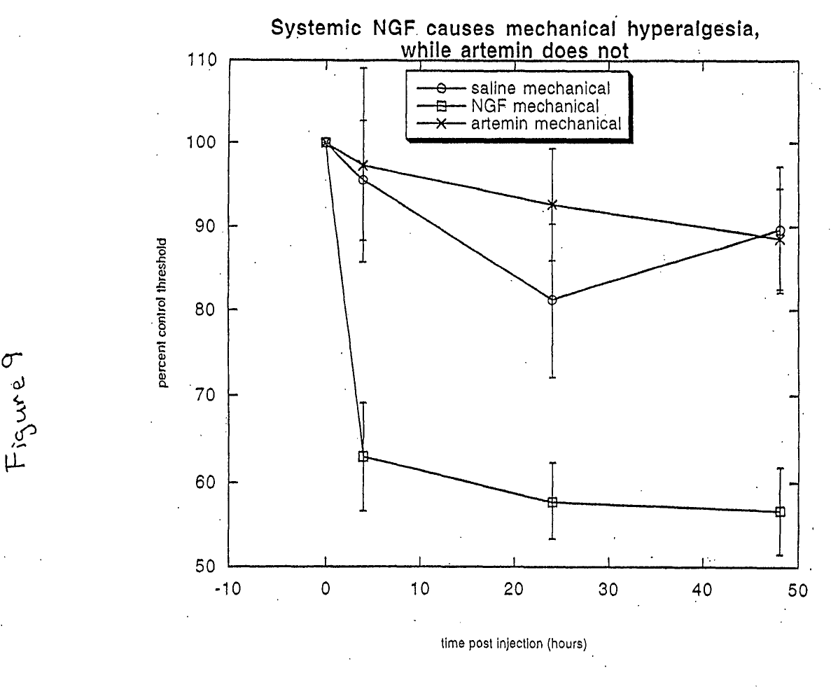

The effects of NGF and artemin on both thermal and mechanical pain thresholds were determined after systemic delivery. Either NGF, artemin or saline was injected subcutaneously into the scruff of female Fischer rats, which had been previously acclimated and tested to determine their withdrawal thresholds to noxious thermal or mechanical stimuli. They were then retested at 2 hdurs, 24 hours or 48 hours post treatment. People blind to the treatment of the animal performed all tests. Thermal withdrawal latencies were measured with the method of Hargreaves, and mechanical thresholds using an electronic version of von Frey hairs. The dose of NGF was 1 mg/kg, and the dose of artemin was 5 mg/kg. The results, presented in FIGS. 8 and 9, clearly show a rapid and prolonged decrease in thermal latency and mechanical threshold in animals treated with NGF, but no significant change in those treated with artemin, as compared to those treated with only saline. FIG. 8 shows that thermal withdrawal latencies in animals treated with NGF dropped to approximately 75% of the pretreatment value, while latencies in animals treated with artemin stayed between 90% and 95% of their pretreatment value. Animals treated with saline showed test latencies of approximately 95% of their pretreatment score. In tests of mechanical sensitivity, animals treated with NGF had their withdrawal thresholds drop to approximately 60% of their pretreatment level, while animals treated with either saline or artemin gradually dropped to about 90% of their pretreatment score (FIG. 9).

### EXAMPLE 2

### Local Administration of NGF Causes Thermal and Mechanical Hyperalgesia While Artemin Does Not

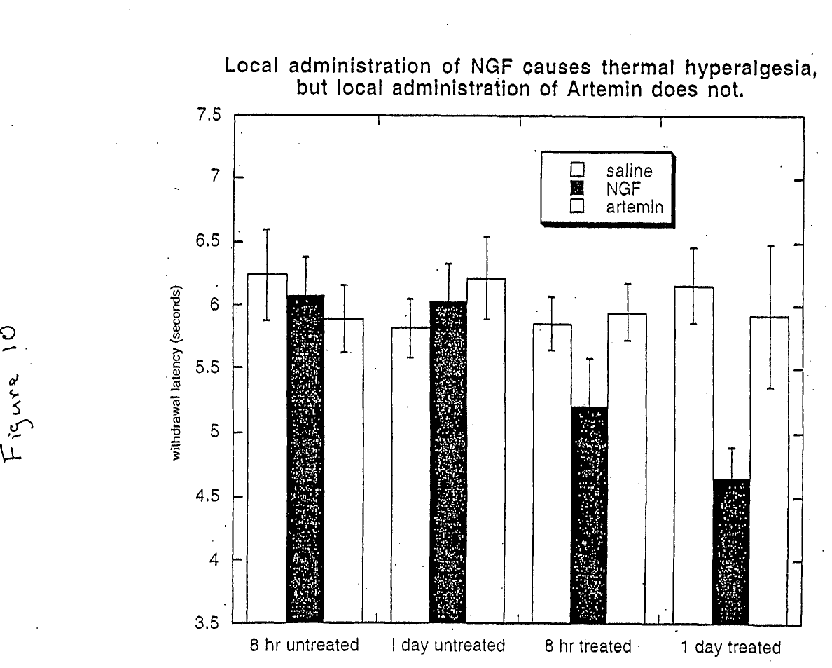

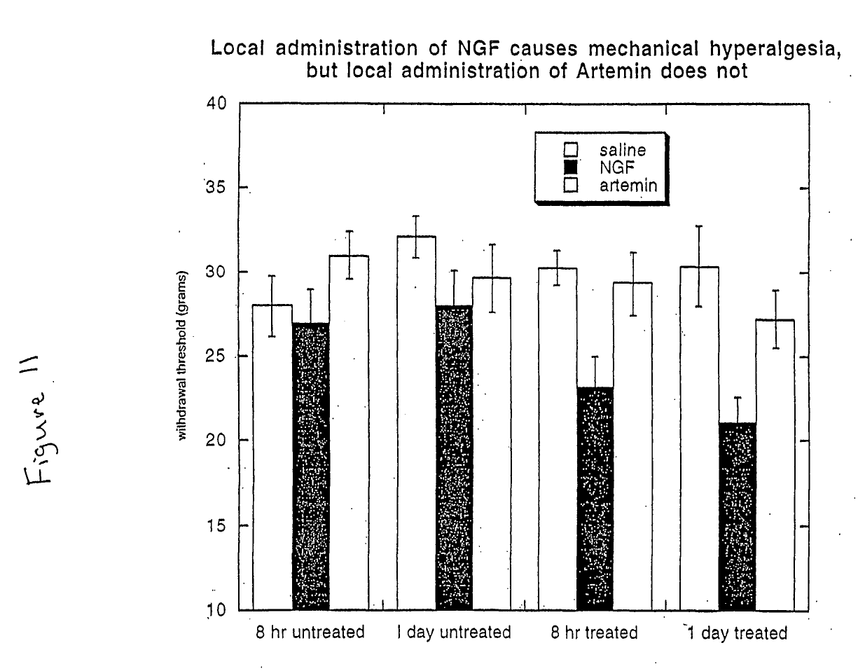

The effects of NGF and artemin on both thermal and mechanical pain thresholds were determined after local delivery. Either NGF, artemin or saline was injected into the plantar surface of one hindpaw of female Fischer rats, which had been previously acclimated and tested to determine their withdrawal thresholds to noxious thermal or mechanical stimuli. They were then retested at 8 hours, 24 hours and 48 hours post treatment. People blind to the treatment of the animal performed all tests. Thermal withdrawal latencies were measured with the method of Hargreaves, and mechanical thresholds using an electronic version of von Frey hairs. The dose of NGF was 1 g and the dose of artemin was 5 g. The results, as presented in FIGS. 10 and 11, clearly show a rapid and prolonged decrease in thermal latency and mechanical threshold in animals treated with NGF, but no significant change in those treated with artemin, as compared to those treated with only saline. Thermal withdrawal latencies in animals treated with NGF dropped from about 6 seconds to approximately 4.5 seconds by 24 hours, while latencies in animals treated with artemin or saline stayed at their pretreatment value of 6 seconds (FIG. 10). In tests of mechanical sensitivity, animals treated with NGF had their withdrawal thresholds drop to approximately 22 grams from a pretreatment value of 27 grams, while the withdrawal threshold did not drop appreciably in animals treated with either saline or artemin (FIG. 11).

### EXAMPLE 3

### Systemic NGF Treatment Causes a Reduction in Body Weight While Artemin Does Not

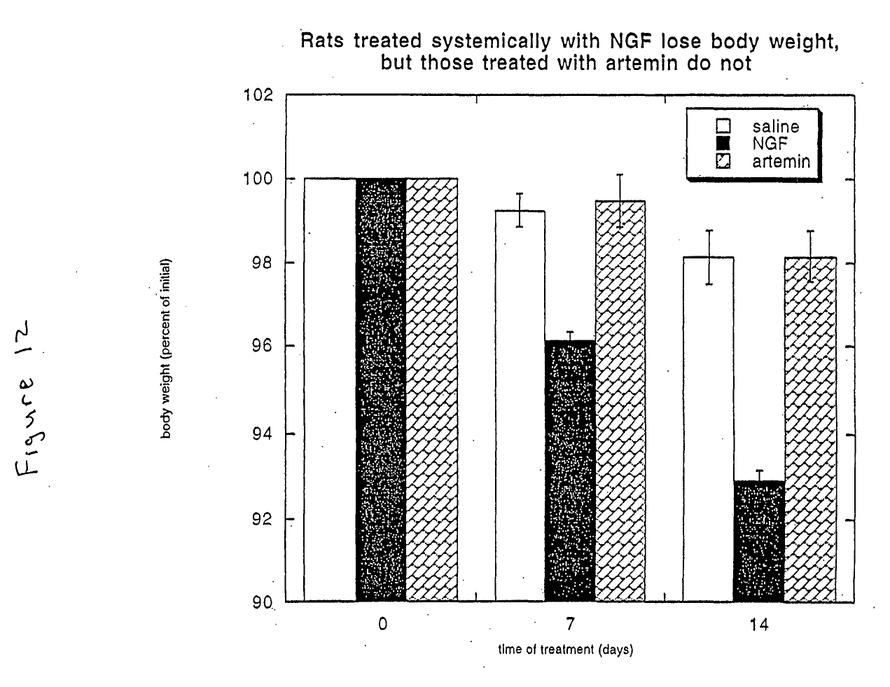

Weight loss was used as a general measure of the chronic pain induced by systemic treatment with NGF, artemin or saline. Female Fischer rats were treated once a week with NGF at 1 mg/kg, artemin at 5 mgikg or saline. Delivery was subcutaneous in the scruff of the neck. Animals were weighed before the first injection and after one and two weeks of treatment. As can be seen in FIG. 12, animals treated with NGF lost approximately 7% of body mass in two weeks, while animals treated with either artemin or saline lost less than 2% of their starting mass.

### EXAMPLE 4

### Artemin Treatment Decreases the Incidence of Pain-Related Behaviors

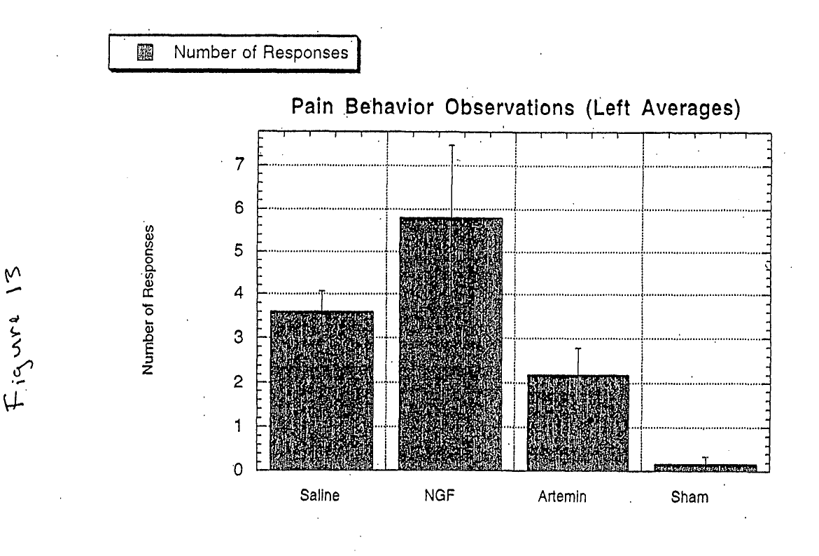

The effects of artemin on neuropathic pain were assessed using the spared nerve injury model in the rat. In this model, the tibial and peroneal nerves are cut and ligated, just distal to the sciatic trifurcation, and the sural and saphenous nerves are left intact. This denervates the top and plantar surface of the hindpaw and leads to a state of long lasting neuropathic pain. Starting immediately after the surgery, animals were treated with intraplantar injections of artemin (5 g), NGF (5 g) or saline, each in 20 I, three times a week. Treated animals were compared to sham animals where the nerves were exposed but not cut or ligated. Spontaneous pain behavior was measured after 12 days of treatment. Animals were placed in a small Plexiglass box and all instances of paw guarding, lifting or flicking were counted for a 5-minute interval. Normal animals or sham surgery animals have very low rates of such behaviors, while spared nerve injury animals display these behaviors relatively frequently. As can be. seen in FIG. 13, constant treatment with NGF increases the frequency of these behaviors twelve days after surgery compared to saline-treated, operated rats. Treatment with artemin, on the other hand, decreases the incidence of these pain-related behaviors (FIG. 13).

### EXAMPLE 5

### Both Artemin and NGF Protect Adult 0RG Neurons From Herpes Simplex Virus Induced Death

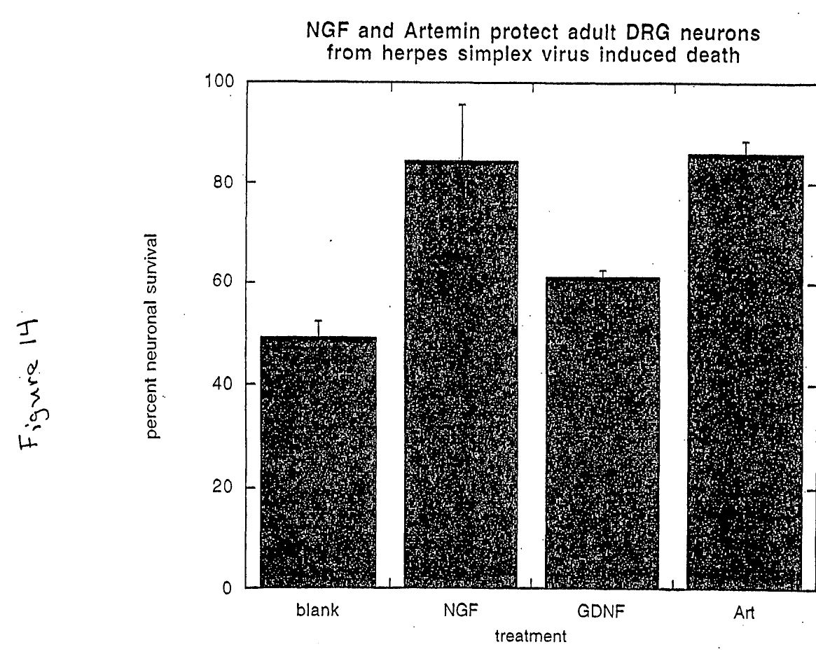

The ability of NGF or artemin to protect neurons from herpes simplex virus induced death was assessed in vitro. Neurons from adult rat dorsal root ganglia were cultured in the presence of NGF (100 ng/ml), artemin (1 g/ml) or basal medium for 11 days and then infected with different concentrations of herpes simplex virus. Surviving cells were counted at two days after infection. FIG. 14 shows that both NGF and artemin protected the neurons from the toxic actions of the virus.

### EXAMPLE 6

### Artemin Treatment Prevents Axotomy-Induced Substance P Loss in the Dorsal Horn of the Spinal Cord

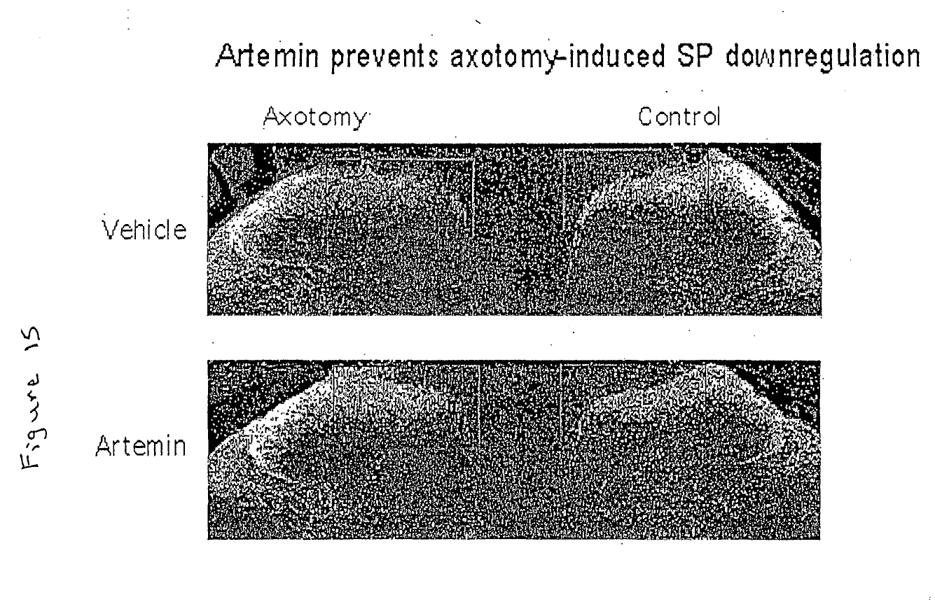

The ability of artemin to protect small fiber sensory neurons from pathological changes associated with injury were tested by examining the effect of artemin administration on the decrease in sensory neuropeptides that normally occurs following axotomy. The central projection of small fiber sensory neurons to the superficial layers of the dorsal horn of the spinal cord can be visualized due to the presence of various neuropeptides contained within the fibers of the sensory neurons. After a transection of the sciatic nerve, the content of peptide in this layer decreases, due to a combination of a withdrawal of the fibers, and a decrease in the peptide content of the fibers. Delivery of artemin (12 g per day) into the intrathecal space essentially completely blocks this decrement. In FIG. 15, sections of spinal cord dorsal horn stained for substance P are shown. There is a decrease in the staining intensity on the side ipsilateral to the axotomy compared to the contralateral side in the saline treated animals, whereas this difference in intensity is essentially abolished on the artemin treated side.

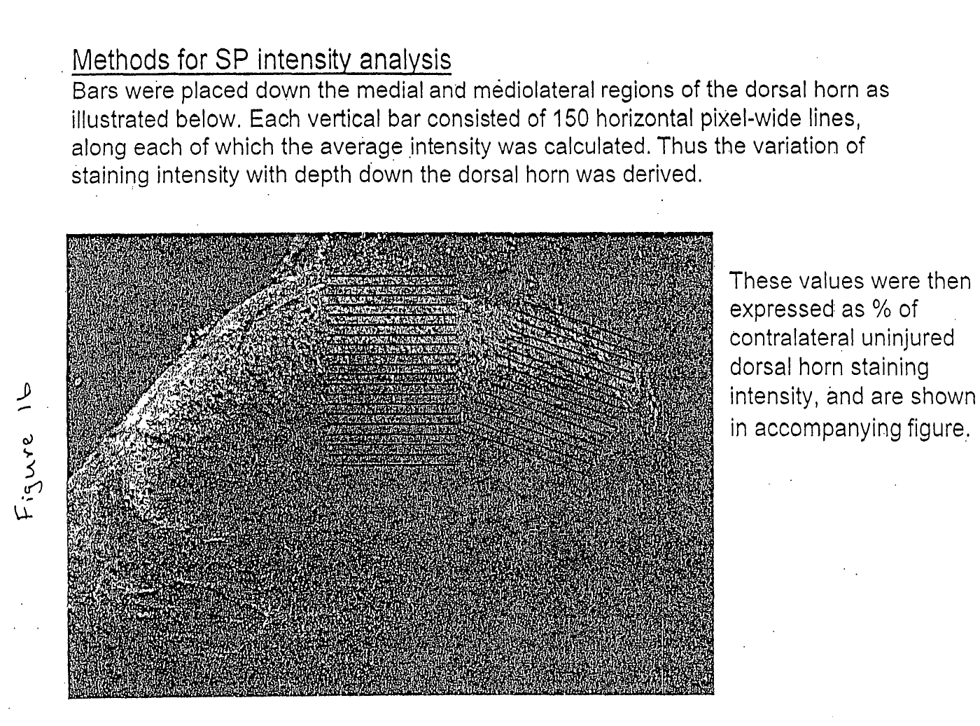

FIG. 16 shows the method of quantifying this result. The average staining intensity is measured in 150, one pixel wide stripes starting at the top of the dorsal horn. Briefly, bars were placed down the medial and mediolateral regions of the dorsal horn as illustrated. Each vertical bar consisted of 150 horizontal pixelwide lines. The average intensity along each line was calculated. Thus the variation in staining intensity with depth down the dorsal horn was derived. These values were then expressed as the % of contralateral uninjured dorsal horn staining intensity and are plotted in FIG. 16.

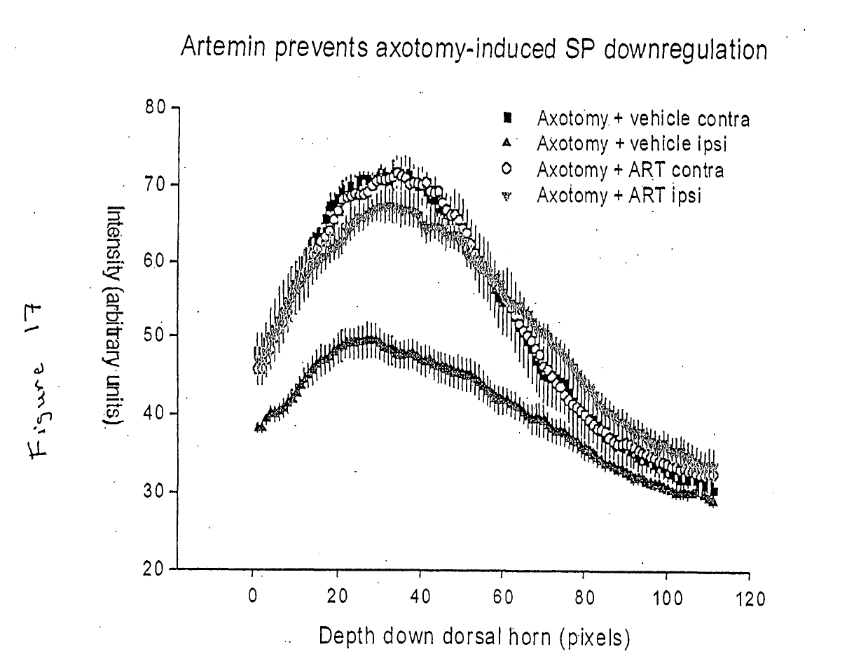

In FIG. 17, intensity of substance P staining (Y-axis) is plotted against depth from the surface of the dorsal horn (X-axis) for rats which had one sciatic nerve cut. On the side of the sciatic injury, there is a significant decrease in substance P immunostaining in the dorsal horn compared to the unoperated side. In animals treated intrathecally with 12 giday of artemin via a continuous infusion, this axotomy-induced peptide loss is prevented.

### EXAMPLE 7

### Artemin Prevents Changes in C-Fiber Conduction Velocity After Axotomy

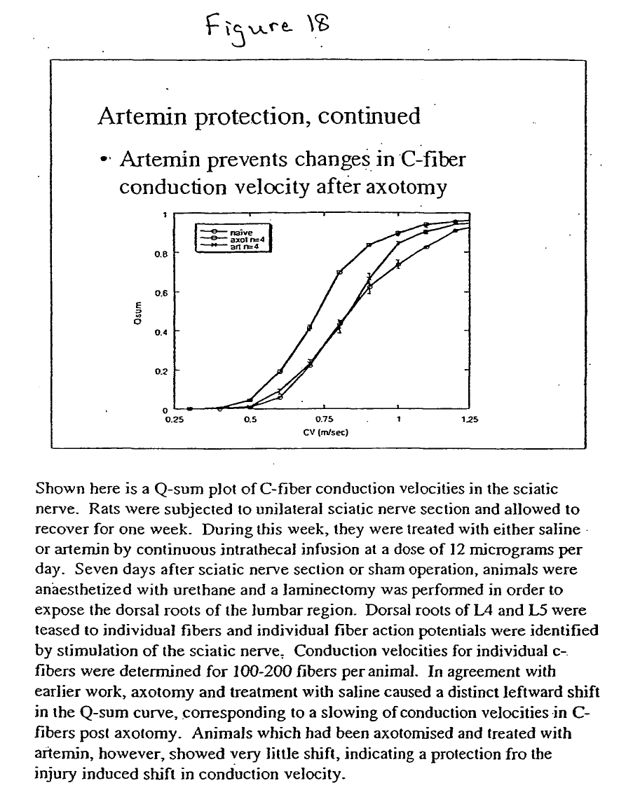

FIG. 18 shows a f-sum plot of C-fiber conduction velocities in the sciatic nerve. Rats were subjected to unilateral sciatic nerve section and allowed to recover for one week. During this week they were treated with either saline or artemin by continuous intrathecal infusion at a dose of 12 giday. Seven days after sciatic nerve section or sham operation, animals were anaesthetized with urethane and a laminectomy was performed in order to expose the dorsal roots of the lumbar region. Dorsal roots of L4 and L5 were teased to individual fibers and individual fiber action potentials were identified by stimulation of the sciatic nerve. Conduction velocities for individual c-fibers were determined for 100-200 fibers per animal. In agreement with earlier work, axotomy and treatment with saline caused a distinct leftward shift in the U-sum curve (FIG. 18), corresponding to a slowing of conduction velocities in C-fibers post axotomy. Animals that had been axotomised and treated with artemin, however, showed very little shift, indicating a protection from the injury induced shift in conduction velocity.

### EXAMPLE 8

### Artemin Induced Survival of Neonatal DRG Neurons Requires GFR 3

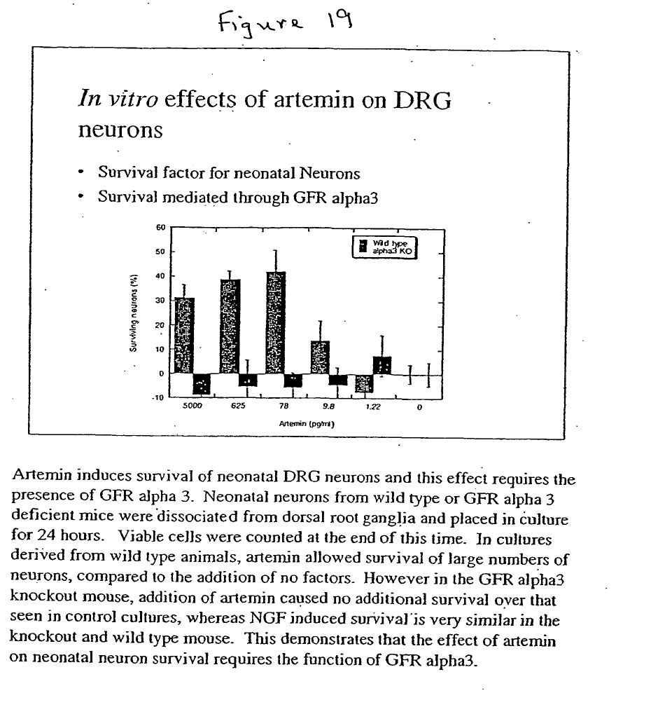

Neonatal neurons from wild type or GFR 3 deficient mice were dissociated from dorsal root ganglia and placed in culture for 24 hours. Viable cells were counted at the end of this time. In cultures derived from wild type animals, artemin allowed survival of large numbers of neurons, compared to the addition of no factors (FIG. 19). As can be seen in FIG. 19, however, in the GFR 3 knockout mouse neurons, addition of artemin caused no additional survival over that seen in control cultures. NGF, on the other hand, induced survival to a similar extent in both the wild type and knockout mouse neurons. This demonstrates that the effect of artemin on neonatal neuron survival requires the function of GFR 3.

### EXAMPLE 9

### GFR 3 is Present on Almost All Small Cells Following Axotomy

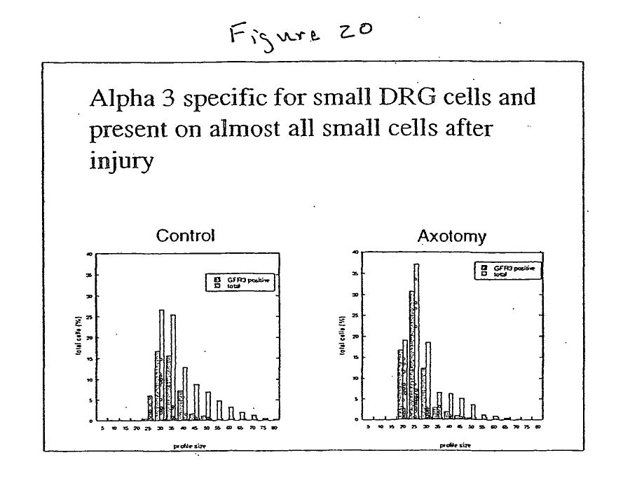

Approximately 40% of dorsal root ganglion (DRG) cells express GFR 3 mRNA. The majority of the ORG cells expressing GFR 3 are small diameter cells. As can be seen in FIG. 20, peripheral nerve injury induces an upregulation in GFR 3 expression so that almost all small DRG cells express the receptor (66% of the total cells). There is a concomitant upregulation of artemin in the distal stump. These data suggest that artemin may have neuroprotective actions on injured small sensory neurons.

### EXAMPLE 10

### Peripherally Delivered Artemin Protects Peptidergic Neurons from Capsaicin

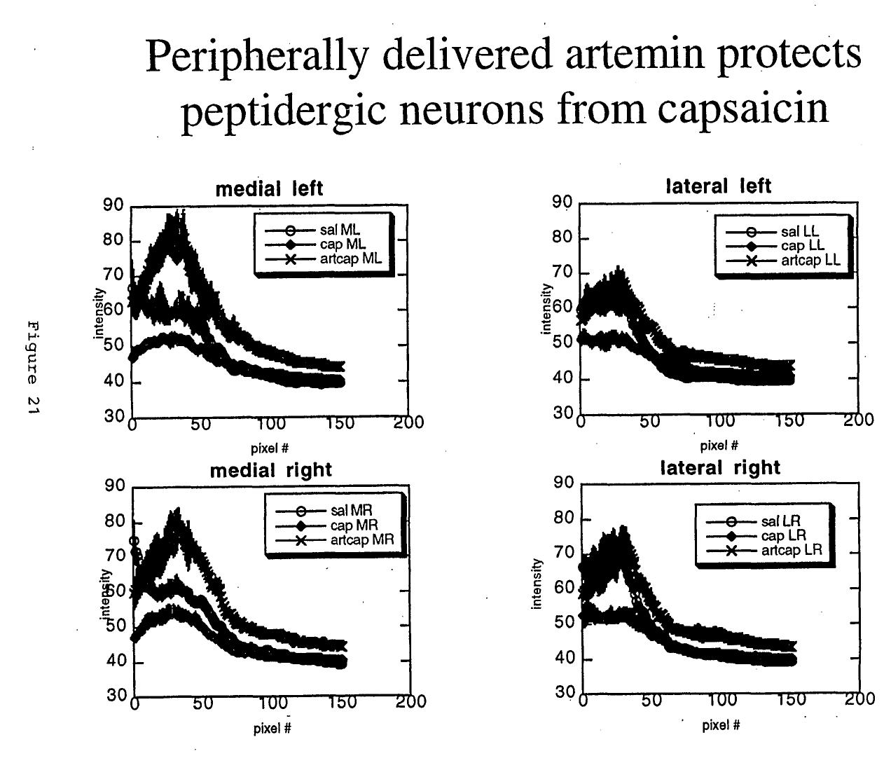

Rats were treated with systemic capsaicin (50 mg/kg) in order to injure small diameter fibers. Some animals were treated every day for 5 days with artemin (5 g delivered intraplantar). After. 5 days, the spinal cords were removed and stained for substance P immunoreactivity in the lateral and medial areas of the dorsal horn. As is shown in FIG. 21, capsaicin treatment caused a profound deficit in the substance P content of both medial and lateral areas of the dorsal horn. The deficit in the lateral dorsal horn was completely blocked by artemin administration into the paw, while artemin administration into the paw gave supranormal levels of substance P in the medial dorsal horn. Thus peripheral artemin administration completely protected the peptide content of the sensory neuron projection into the dorsal horn from the deleterious effects of capsaicin.

### EXAMPLE 11

### ICV Artemin Prevents Loss of Dorsal Horn Substance P After Sciatic Axotomy

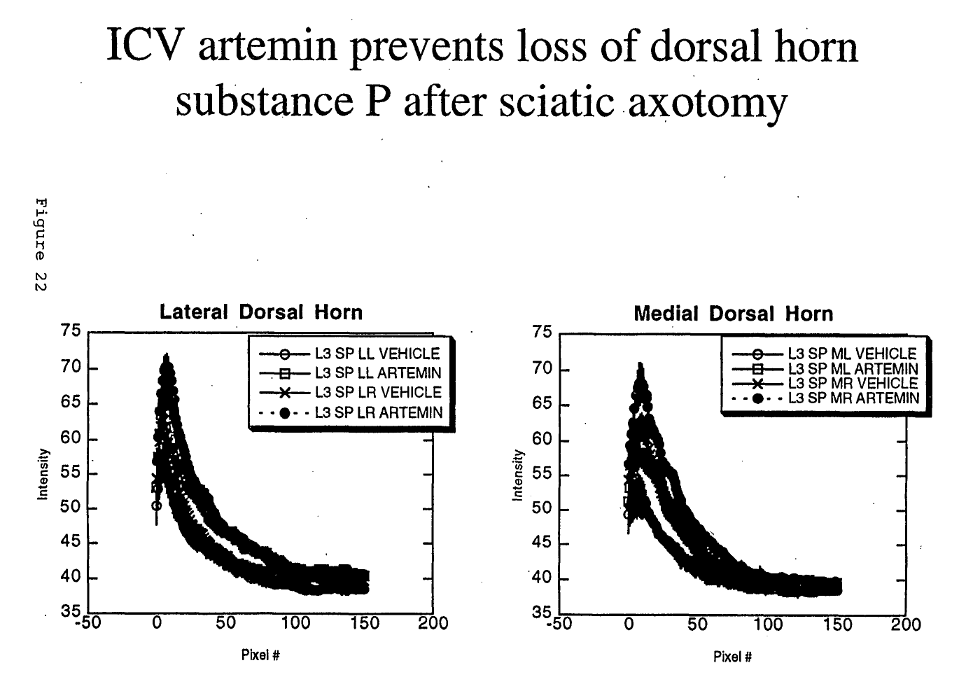

Mice were implanted with cannula into the lateral ventricle and the left sciatic-nerve was cut. Either artemin or vehicle was infused via the cannula with an osmotic minipump at a rate of 12 g/day. After 8 days, the spinal cords were removed and stained for substance P immunoreactivity in the lateral and medial areas of the dorsal horn. FIG. 22 shows that axotomized animals treated with vehicle showed a loss of staining intensity for substance P on the lesion side (blue) compared to the control side (green). This effect was most pronounced in the medial dorsal horn, in keeping with the known projection pattern of the sciatic nerve. Artemin infusion largely prevented this loss of substance P on the lesion side (red) and caused an increase of peptide staining on the control side (black).

1. A method for the prevention or treatment of pain comprising administering to a subject in need an effective amount of artemin or an agonist thereof.

2. The method of claim 1 wherein said pain is chronic pain.

3. The method of claim 1 wherein said pain is neuropathic pain.

4. The method of claim 1 wherein said pain is associate with nerve injury.

5. The method of claim 1 wherein said artemin is a native-sequence artemin polypeptide.

6. The method of claim 5 wherein said artemin is a native-sequence human artemin polypeptide.

7. The method of claim 6 wherein said subject is a human patient.

8. The method of claim 7 wherein said artemin comprises a polypeptide of SEQ ID NO: 1, SEQ ID NO: 3, SEQ ID NO. 5 or a biologically active fragment thereof.

9. The method of claim 7 wherein said artemin is an immunoadhesin.


SEQUENCE LISTING (7 sequences)

SEQ ID NO: 1 - protein, 113 aa, Homo Sapiens
AGGPGSRARAAGARGCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 2 - DNA, 663 bp, Homo Sapiens
atggaacttggacttggaggcctctccacgctgtcccactgcccctggcctaggcggcagcctgccctgtggcccaccctggccgctctggctctgctgagcagcgtcgcagaggcctccctgggctccgcgccccgcagccctgccccccgcgaaggccccccgcctgtcctggcgtcccccgccggccacctgccggggggacgcacggcccgctggtgcagtggaagagcccggcggccgccgccgcagccttctcggcccgcgcccccgccgcctgcacccccatctgctcttccccgcgggggccgcgcggcgcgggctgggggcccgggcagccgcgctcgggcagcgggggcgcggggctgccgcctgcgctcgcagctggtgccggtgcgcgcgctcggcctgggccaccgctccgacgagctggtgcgtttccgcttctgcagcggctcctgccgccgcgcgcgctctccacacgacctcagcctggccagcctactgggcgccggggccctgcgaccgcccccgggctcccggcccgtcagccagccctgctgccgacccacgcgctacgaagcggtctccttcatggacgtcaacagcacctggagaaccgtggaccgcctctccgccaccgcctgcggctgcctgggctga

SEQ ID NO: 3 - protein, 220 aa, Homo Sapiens
MELGLGGLSTLSHCPWPRRQPALWPTLAALALLSSVAEASLGSAPRSPAPREGPPPVLASPAGHLPGGRTARWCSGRARRPPPQPSRPAPPPPAPPSALPRGGRAARAGGPGSRARAAGARGCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 4 - DNA, 714 bp, Homo Sapiens
atgcccggcctgatctcagcccgaggacagcccctccttgaggtccttcctccccaagcccacctgggtgccctctttctccctgaggctccacttggtctctccgcgcagcctgccctgtggcccaccctggccgctctggctctgctgagcagcgtcgcagaggcctccctgggctccgcgccccgcagccctgccccccgcgaaggccccccgcctgtcctggcgtcccccgccggccacctgccggggggacgcacggcccgctggtgcagtggaagagcccggcggccgccgccgcagccttctcggcccgcgcccccgccgcctgcacccccatctgctcttccccgcgggggccgcgcggcgcgggctgggggcccgggcagccgcgctcgggcagcgggggcgcggggctgccgcctgcgctcgcagctggtgccggtgcgcgcgctcggcctgggccaccgctccgacgagctggtgcgtttccgcttctgcagcggctcctgccgccgcgcgcgctctccacacgacctcagcctggccagcctactgggcgccggggccctgcgaccgcccccgggctcccggcccgtcagccagccctgctgccgacccacgcgctacgaagcggtctccttcatggacgtcaacagcacctggagaaccgtggaccgcctctccgccaccgcctgcggctgcctgggctga

SEQ ID NO: 5 - protein, 237 aa, Homo Sapiens
MPGLISARGQPLLEVLPPQAHLGALFLPEAPLGLSAQPALWPTLAALALLSSVAEASLGSAPRSPAPREGPPPVLASPAGHLPGGRTARWCSGRARRPPPQPSRPAPPPPAPPSALPRGGRAARAGGPGSRARAAGARGCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 6 - DNA, 675 bp, Murine
atggaactgggacttgcagagcctactgcattgtcccactgcctccggcctaggtggcagtcagcctggtggccaaccctagctgttctagccctgctgagctgcgtcacagaagcttccctggacccaatgtcccgcagccccgccgctcgcgacggtccctcaccggtcttggcgccccccacggaccacctgcctgggggacacactgcgcatttgtgcagcgaaagaaccctgcgacccccgcctcagtctcctcagcccgcacccccgccgcctggtcccgcgctccagtctcctcccgctgcgctccgcggggcacgcgcggcgcgtgcaggaacccggagcagccgcgcacggaccacagatgcgcgcggctgccgcctgcgctcgcagctggtgccggtgagtgcgctcggcctaggccacagctccgacgagctgatacgtttccgcttctgcagcggctcgtgccgccgagcacgctcccagcacgatctcagtctggccagcctactgggcgctggggccctacggtcgcctcccgggtcccggccgatcagccagccctgctgccggcccactcgctatgaggccgtctccttcatggacgtgaacagcacctggaggaccgtggaccacctctccgccactgcctgcggctgtctgggctga

SEQ ID NO: 7 - protein, 224 aa, Murine
MELGLAEPTALSHCLRPRWQSAWWPTLAVLALLSCVTEASLDPMSRSPAARDGPSPVLAPPTDHLPGGHTAHLCSERTLRPPPQSPQPAPPPPGPALQSPPAALRGARAARAGTRSSRARTTDARGCRLRSQLVPVSALGLGHSSDELIRFRFCSGSCRRARSQHDLSLASLLGAGALRSPPGSRPISQPCCRPTRYEAVSFMDVNSTWRTVDHLSATACGCLG